SEQUENCE LISTING (74 sequences)

SEQ ID NO: 1 - DNA, 29 bp, Artificial sequence
  synthetic oligonucleotide primer
cccaagcttcgtcgtagcgttccagcagc

SEQ ID NO: 2 - DNA, 30 bp, Artificial sequence
  synthetic oligonucleotide primer
ccggaattcgagacccagagccagcataca

SEQ ID NO: 3 - DNA, 31 bp, Artificial sequence
  synthetic oligonucleotide primer
ccggaattcttcctggaggacttcctggtgg

SEQ ID NO: 4 - DNA, 30 bp, Artificial sequence
  synthetic oligonucleotide primer
tgctctagacggtgaagaaggtgaggtcgc

SEQ ID NO: 5 - DNA, 23 bp, Artificial sequence
  synthetic oligonucleotide primer
acatttcgtagtgctggtgcttg

SEQ ID NO: 6 - DNA, 21 bp, Artificial sequence
  synthetic oligonucleotide primer
gatggtgtccacgtcctcgtc

SEQ ID NO: 7 - DNA, 27 bp, Artificial sequence
  synthetic oligonucleotide primer
atcatatgctggctctgggtctcggcg

SEQ ID NO: 8 - DNA, 27 bp, Artificial sequence
  synthetic oligonucleotide primer
cggaattctcactcggtctcctccgtc

SEQ ID NO: 9 - DNA, 27 bp, Artificial sequence
  synthetic oligonucleotide primer
cccaagcttgctgaagggctccaccga

SEQ ID NO: 10 - DNA, 27 bp, Artificial sequence
  synthetic oligonucleotide primer
ccggaattcggccgaaatcccgtccac

SEQ ID NO: 11 - DNA, 27 bp, Artificial sequence
  synthetic oligonucleotide primer
ccggaattcgtcggccggttcagctga

SEQ ID NO: 12 - DNA, 27 bp, Artificial sequence
  synthetic oligonucleotide primer
tgctctagacaggagctgaaggagatc

SEQ ID NO: 13 - DNA, 22 bp, Artificial sequence
  synthetic oligonucleotide primer
gcgacggacagaaccgcctcaa

SEQ ID NO: 14 - DNA, 22 bp, Artificial sequence
  synthetic oligonucleotide primer
acgccctgctggtctccttcca

SEQ ID NO: 15 - DNA, 31 bp, Artificial sequence
  synthetic oligonucleotide primer
cgcatatgatatccgtggacgggatttcggc

SEQ ID NO: 16 - DNA, 27 bp, Artificial sequence
  synthetic oligonucleotide primer
tagaattctcagctgaaccggccgacg

SEQ ID NO: 17 - DNA, 29 bp, Artificial sequence
  synthetic oligonucleotide primer
cccaagcttgctcggcgtagtaggggtcc

SEQ ID NO: 18 - DNA, 29 bp, Artificial sequence
  synthetic oligonucleotide primer
ccggaattctccccagacgccttgctgta

SEQ ID NO: 19 - DNA, 29 bp, Artificial sequence
  synthetic oligonucleotide primer
ccggaattccccaacaccgagtaccacga

SEQ ID NO: 20 - DNA, 29 bp, Artificial sequence
  synthetic oligonucleotide primer
tgctctagacgtcgcttccatcttcgtca

SEQ ID NO: 21 - DNA, 20 bp, Artificial sequence
  synthetic oligonucleotide primer
tgaagcccagcagctccacc

SEQ ID NO: 22 - DNA, 21 bp, Artificial sequence
  synthetic oligonucleotide primer
cgaccaggatgacctcgaacc

SEQ ID NO: 23 - DNA, 27 bp, Artificial sequence
  synthetic oligonucleotide primer
atcatatggccccattgcgtgggagat

SEQ ID NO: 24 - DNA, 34 bp, Artificial sequence
  synthetic oligonucleotide primer
atggatccaattgtcaaacgcgcgtgaactggtg

SEQ ID NO: 25 - DNA, 29 bp, Artificial sequence
  synthetic oligonucleotide primer
cccaagctttcggggagatcgccctggaa

SEQ ID NO: 26 - DNA, 29 bp, Artificial sequence
  synthetic oligonucleotide primer
ccggaattccaccttctcgtagaacaggc

SEQ ID NO: 27 - DNA, 29 bp, Artificial sequence
  synthetic oligonucleotide primer
ccggaattcgtcagcgaccgcaaggagga

SEQ ID NO: 28 - DNA, 29 bp, Artificial sequence
  synthetic oligonucleotide primer
tgctctagagtggatcaggacgtcacggg

SEQ ID NO: 29 - DNA, 20 bp, Artificial sequence
  synthetic oligonucleotide primer
ccttctggcggaacctgctc

SEQ ID NO: 30 - DNA, 20 bp, Artificial sequence
  synthetic oligonucleotide primer
gtggatcaggacgtcacggg

SEQ ID NO: 31 - DNA, 30 bp, Artificial sequence
  synthetic oligonucleotide primer
tacatatggctgacgcggtgctgctgacgc

SEQ ID NO: 32 - DNA, 28 bp, Artificial sequence
  synthetic oligonucleotide primer
atgaattctcatccggccagggcccgtg

SEQ ID NO: 33 - DNA, 27 bp, Artificial sequence
  synthetic oligonucleotide primer
cccaagcttgctacaaccacgccgtgg

SEQ ID NO: 34 - DNA, 27 bp, Artificial sequence
  synthetic oligonucleotide primer
ccggaattcgggtgtcagcaggaccac

SEQ ID NO: 35 - DNA, 27 bp, Artificial sequence
  synthetic oligonucleotide primer
ccggaattcctgatcaaccgggccatc

SEQ ID NO: 36 - DNA, 27 bp, Artificial sequence
  synthetic oligonucleotide primer
tgctctagagcggaacggcatgttcac

SEQ ID NO: 37 - DNA, 23 bp, Artificial sequence
  synthetic oligonucleotide primer
cttctgctacgtgccgcgggccc

SEQ ID NO: 38 - DNA, 25 bp, Artificial sequence
  synthetic oligonucleotide primer
gcggtcagcgagtggcagcggaacg

SEQ ID NO: 39 - DNA, 30 bp, Artificial sequence
  synthetic oligonucleotide primer
ttcatatgtcggacgtggtcctgctgacac

SEQ ID NO: 40 - DNA, 28 bp, Artificial sequence
  synthetic oligonucleotide primer
atgaattctcagcccctcacctgtgcca

SEQ ID NO: 41 - DNA, 27 bp, Artificial sequence
  synthetic oligonucleotide primer
cccaagcttgcgcacaccatgtaccag

SEQ ID NO: 42 - DNA, 27 bp, Artificial sequence
  synthetic oligonucleotide primer
ccggaattcgagggtgggggtgtgcat

SEQ ID NO: 43 - DNA, 27 bp, Artificial sequence
  synthetic oligonucleotide primer
ccggaattcctgatgcgcggcctgcac

SEQ ID NO: 44 - DNA, 27 bp, Artificial sequence
  synthetic oligonucleotide primer
tgctctagagctcgcctactacgacag

SEQ ID NO: 45 - DNA, 22 bp, Artificial sequence
  synthetic oligonucleotide primer
cggcggcgacctcaccttcttc

SEQ ID NO: 46 - DNA, 22 bp, Artificial sequence
  synthetic oligonucleotide primer
ccccggaacagccgcaggacgg

SEQ ID NO: 47 - DNA, 30 bp, Artificial sequence
  synthetic oligonucleotide primer
acatgcatgcaatgggttcatgtgcagctc

SEQ ID NO: 48 - DNA, 30 bp, Artificial sequence
  synthetic oligonucleotide primer
acatgcatgcagcatatcatcagcgagctg

SEQ ID NO: 49 - DNA, 32 bp, Artificial sequence
  synthetic oligonucleotide primer
gaagatctgcatatgagcggccctggttacct

SEQ ID NO: 50 - DNA, 29 bp, Artificial sequence
  synthetic oligonucleotide primer
ggaattctcatttcctcgcaaccacttcg

SEQ ID NO: 51 - DNA, 23 bp, Artificial sequence
  synthetic oligonucleotide primer
tcccgctcgctgttccggtacta

SEQ ID NO: 52 - DNA, 22 bp, Artificial sequence
  synthetic oligonucleotide primer
tgtccacgtcctcgtcggtcat

SEQ ID NO: 53 - DNA, 27 bp, Artificial sequence
  synthetic oligonucleotide primer
cccaagctttccttgaggtgcagcgcg

SEQ ID NO: 54 - DNA, 27 bp, Artificial sequence
  synthetic oligonucleotide primer
ccggaattcgtcgcgatgtgcgaacat

SEQ ID NO: 55 - DNA, 27 bp, Artificial sequence
  synthetic oligonucleotide primer
ccggaattccgtctgcgggagctgaag

SEQ ID NO: 56 - DNA, 27 bp, Artificial sequence
  synthetic oligonucleotide primer
tgctctagaagctcctcgacgaagaac

SEQ ID NO: 57 - DNA, 19 bp, Artificial sequence
  synthetic oligonucleotide primer
gttggcgaccgagttgagc

SEQ ID NO: 58 - DNA, 21 bp, Artificial sequence
  synthetic oligonucleotide primer
gaacccgtagtagtgcgtggc

SEQ ID NO: 59 - DNA, 27 bp, Artificial sequence
  synthetic oligonucleotide primer
cccaagcttgacttcgactacgtcctc

SEQ ID NO: 60 - DNA, 27 bp, Artificial sequence
  synthetic oligonucleotide primer
ccggaattcgaccacgctcaccgagat

SEQ ID NO: 61 - DNA, 29 bp, Artificial sequence
  synthetic oligonucleotide primer
ccggaattcgacgtcggcaccttcctcgc

SEQ ID NO: 62 - DNA, 29 bp, Artificial sequence
  synthetic oligonucleotide primer
tgctctagagaggccgcacagcagccatt

SEQ ID NO: 63 - DNA, 23 bp, Artificial sequence
  synthetic oligonucleotide primer
caacaccgagtaccacgacaagc

SEQ ID NO: 64 - DNA, 21 bp, Artificial sequence
  synthetic oligonucleotide primer
gtcgccgtcccactccggttc

SEQ ID NO: 65 - DNA, 32 bp, Artificial sequence
  synthetic oligonucleotide primer
gaggatccgcatatgccggagggtacggccgg

SEQ ID NO: 66 - DNA, 31 bp, Artificial sequence
  synthetic oligonucleotide primer
accatgggcagcatgccgcccgcgccggcca

SEQ ID NO: 67 - DNA, 32 bp, Artificial sequence
  synthetic oligonucleotide primer
ggaattcgcggcatgctgctggtggggctctc

SEQ ID NO: 68 - DNA, 25 bp, Artificial sequence
  synthetic oligonucleotide primer
ccaggggaaggccatggtcggtacg

SEQ ID NO: 69 - DNA, 25 bp, Artificial sequence
  synthetic oligonucleotide primer
cgtaccgaccatggccttcccctgg

SEQ ID NO: 70 - DNA, 30 bp, Artificial sequence
  synthetic oligonucleotide primer
ggaattctcaggggcgggacgtggtgacca

SEQ ID NO: 71 - DNA, 24 bp, Artificial sequence
  synthetic oligonucleotide primer
atcagcgatgtgttcgcgatcatc

SEQ ID NO: 72 - DNA, 22 bp, Artificial sequence
  synthetic oligonucleotide primer
acgttgatgcaggtggacagcc

SEQ ID NO: 73 - DNA, 18 bp, Artificial sequence
  synthetic oligonucleotide primer
gagcccgtcgcgatcgtc

SEQ ID NO: 74 - DNA, 20 bp, Artificial sequence
  synthetic oligonucleotide primer
cgcttcttcgaggatcatgt

(19) United States
(12) Patent Application Publication
(10) Pub. No.: US 2013/0231377 A1
(43) Pub. Date: Sep. 5, 2013

(54) PACTAMYCIN ANALOGS AND METHODS OF USE

(71) Applicant: State of Oregon acting by and through the state Board of Higher Education on behalf of, Oregon State University, Corvalllis, OR (US)

(72) Inventor: Taifo Mahmud, Corvallis, OR (US)

(73) Assignees: Oregon State University, Corvallis, OR (US); State of Oregon acting by and through the state Board of Higher Education on behalf of

(21) Appl. No.: 13/813,144

(22) PCT Filed: Aug. 2, 2011
(86) PCT No.: PCT/US11/46322

§ 371 Date: Jan. 29, 2013

Related U.S. Application Data
(60) Provisional application No. 61/370,094, filed on Aug. 2, 2010.

(51) Int. Cl. C07C 275/26; C07D 263/52; C07D 235/02

(52) U.S. Cl. CPC C07C 275/26; C07D 235/02; C07D 263/52
USPC 514/376; 548/302.7; 514/387; 560/70; 514/534; 564/47; 514/595; 548/216

(57) ABSTRACT

Disclosed are pactamycin analogs, pharmaceutical compositions including the analogs, and methods of using the analogs, such as to inhibit tumor growth or a pathogenic infection such as a bacterial or parasitic infection. The pactamycin analogs have a general formula

where R¹ is H, lower aliphatic, amide, acyl, or aminoacyl; R² is —C(O)NR⁸R⁹ where R⁸ and R⁹ independently are hydrogen or lower aliphatic, or R¹ and R² together form a cyclic structure; R³ and R⁴ independently are hydrogen, hydroxyl, or lower aliphatic, or R² and R³ together form a cyclic structure; R⁵ is hydrogen or acyl; R⁶ and R⁷ independently are hydrogen, hydroxyl, halogen, lower aliphatic, or amino.

### CROSS-REFERENCE TO RELATED APPLICATION

This application claims the benefit of the earlier filing date of U.S. Provisional Application No. 61/370,094, filed Aug. 2, 2010, which is incorporated in its entirety herein by reference.

### FIELD

This disclosure relates to pactamycin analogs and methods of use thereof, including use as antimicrobial or antitumor agents.

### BACKGROUND

The global emergence of multidrug-resistant bacterial infections has resulted in enormous healthcare costs and has become a major threat to public health. In the U.S. alone, the total cost linked to antibiotic-resistant infections has been estimated at $5 billion annually (Zinner, Expert Rev. Anti. Infect. Ther., 3: 907-913, 2005). About 70% of the bacteria that cause infections in hospitals are now resistant to at least one of the drugs most commonly used for treatment. For example, some organisms are resistant to all approved antibiotics and can be treated only with experimental and potentially toxic drugs. Pactamycin, an aminocyclitol antibiotic, has pronounced antibacterial, antitumor, antiviral, and antiplasmodial activities. However, its development as a clinical drug has been hampered by its broad cytotoxicity.

Therefore, to stay ahead of the development of antibacterial drug resistance, there is a pressing necessity to identify new antibiotics, especially those with novel mechanisms of action and reduced mammalian cytotoxicity, and methods for producing such antibiotics.

### SUMMARY

Disclosed herein are pactamycin analogs. The disclosed analogs have antimicrobial activities as well as other useful pharmaceutical activities including antitumor activities. For examples, the analogs include at least one of antibacterial, antitumor, or antimalarial activities.

In some embodiments, a disclosed pactamycin analog, other than pactamycin, 7-deoxypactamycin, or pactamycate, has a chemical structure according to general formula (I)

where R¹ is H, lower aliphatic, amide, acyl, or aminoacyl; R² is —C(O)NR⁸R⁹ where R⁸ and R⁹ independently are hydrogen or lower aliphatic, or R¹ and R² together form a cyclic structure; R³ and R⁴ independently are hydrogen, hydroxyl, or lower aliphatic, or R² and R³ together form a cyclic structure; R⁵ is hydrogen or acyl; R⁶ and R⁷ independently are hydrogen, hydroxyl, halogen, lower aliphatic, or amino.

In some embodiments, R³ and R⁴ are hydrogen, and the pactamycin analog has a chemical structure according to general formula (II)

In certain embodiments, R⁵ is —C(O)R¹⁰, and R¹⁰ is

wherein X is selected from H, lower alkyl, —OR¹¹, halogen, —NO₂, —NR¹²R¹³; Y is H, halogen or lower alkyl; and R¹¹, R¹² and R¹³ independently are selected from H, lower alkyl and acyl. In particular embodiments, R⁵ is a substituted benzoyl group, such as 2-hydroxy-6-methylbenzoyl.

In one embodiment, R⁶ is hydrogen, hydroxyl, lower aliphatic, or amino. In one embodiment, R⁷ is hydrogen, halogen, lower aliphatic, or amino.

In one embodiment, R¹ and R² together form a heterocyclic 5-membered ring, such as an imidazolidin-2-one ring. In one embodiment, R² and R³ together form a heterocyclic 5-membered ring, e.g., a carbamate-containing ring such as a 1,3-oxazolidin-2-one ring.

Exemplary pactamycin analogs have a formula selected from

Also disclosed are pharmaceutical compositions. In some examples, a pharmaceutical composition includes a pactamycin analog and a pharmaceutically acceptable carrier.

Methods of use of the disclosed pactamycin analogs are also provided. For example, methods of treating a pathogen of interest (such as a bacteria or a parasite) in a subject with one or more of the disclosed pactamycin analogs are provided. In one particular example, a method of treating a subject with a parasitic infection is provided. Methods of inhibiting or preventing tumor formation in a subject with one or more of the disclosed pactamycin analogs are also provided. Methods of producing and isolating pactamycin analogs are further disclosed.

The foregoing and other features of the disclosure will become more apparent from the following detailed description, which proceeds with reference to the accompanying figures.

### BRIEF DESCRIPTION OF THE FIGURES

FIG. 1 is a proposed biosynthetic pathway to pactamycin.

FIG. 2 is a series of HPLC spectra of ethyl acetate extracts from the culture broths of S. pactum mutants: A—wild-type, B—ΔptmH, C—ΔptmH/ΔptmQ, D—ΔptmD, E—ΔptmB, F—ΔptmL, G—ΔptmM. Identified peaks included: 1—pactamycin, 2—7-deoxypactamycin, 3—pactamycate, 4—7-demethyl-7-deoxypactamycin, 5—de-6MSA-7-demethyl-7-deoxypactamycin, 6—N,N-didemethylpactamycin, 7—N,N-didemethyl-7-deoxypactamycin.

FIG. 3 is a series of ESI-MS/MS spectra of pactamycin analogs: H—pactamycin, I—7-deoxypactamycin, J—N,N-didemethylpactamycin, K—N,N-didemethyl-7-deoxypactamycin, L—7-demethyl-7-deoxypactamycin, M—de-6MSA-7-demethyl-7-deoxypactamycin.

FIGS. 4A-4B illustrate the fragmentation patterns of pactamycin (FIG. 4A) and N,N-didemethylpactamycin (FIG. 4B).

FIGS. 5A-5E are partial mass spectra of pactamycin analogs extracted from the culture broths of S. pactum mutants: FIG. 5A—wild-type, FIG. 5B—ΔptmD, FIG. 5C—ΔptmH, FIG. 5D—ΔptmQ, FIG. 5E—ΔptmH/ΔptmQ. Thick arrows indicate positions at which modifications take place.

FIGS. 6A-6D are 1D and 2D NMR spectra of 7-demethyl-7-deoxypactamycin: FIG. 6A—¹H NMR spectrum, FIG. 6B—¹³C NMR spectrum, FIG. 6C—HMBC spectrum, FIG. 6D—HSQC spectrum; x, CH₃COONH₄.

FIGS. 7A-7D are 1D and 2D NMR spectra of de-6MSA-7-demethyl-7-deoxypactamycin: FIG. 7A—¹H NMR spectrum, FIG. 7B—¹³C NMR spectrum, FIG. 7C—HMBC spectrum, FIG. 7D—HSQC spectrum; x, CH₃COONH₄.

FIGS. 8A-8F are dose response curves illustrating the antimalarial activity of two pactamycin analogs against chloroquine-sensitive (D6) and chloroquine-resistant (Dd2) strains of Plasmodium falciparum: (FIG. 8A) chloroquine, (FIG. 8B) de-6MSA-7-demethyl-7-deoxypactamycin, (FIG. 8C) 7-demethyl-7-deoxypactamycin, (FIG. 8D) pactamycate, (FIG. 8E) pactamycin, (FIG. 8F) N-acetylglucosaminyl-3-aminoacetophenone. All values are presented as relative fluorescence units (RFU). Each value is mean±SEM of quadruplicate values from a representative study. The 50% and 90% inhibitory concentrations (IC₅₀ and IC₉₀) were determined by non-linear regression analysis of logistic dose-response curves (GraphPad Prism® software).

FIGS. 9A-9D are digital images of an agar-diffusion assay of two pactamycin analogs against S. aureus (FIG. 9A), B. subtilis (FIG. 9B), P. aeruginosa (FIG. 9C), and E. coli (FIG. 9D). 1-20 μL 10 mM de-6MSA-7-demethyl-7-deoxypactamycin; 2-20 μL 1 mM de-6MSA-7-demethyl-7-deoxypactamycin; 3-20 μL 10 mM 7-demethyl-7-deoxypactamycin; 4-20 μL 1 mM 7-demethyl-7-deoxypactamycin; 5-20 μL 10 mM pactamycin; 6-20 μL 1 mM pactamycin; 7-20 μL 10 mM N-acetyl-glucosaminyl-3-aminoacetophenone; 8-5 μL 1 mg/mL ampicillin.

FIGS. 10A-10C are graphs of cytotoxicity assays of two pactamycin analogs against HCT116 cells using broad-range concentrations at 48 hours (FIG. 10A), narrow-range concentrations at 24 hours (FIG. 10B); and narrow-range concentrations at 48 hours (FIG. 10C). All values are presented as percentage of viable cells, which was calculated relative to the “No Treatment” and “Solvent Only” wells. Each value is the mean±SEM of triplicate values from a representative study.

FIG. 11 is a proposed detailed biosynthetic pathway to pactamycin. The dashed arrow shows a proposed shunt pathway to 7-deoxypactamycin.

FIG. 12 is a scheme illustrating the proposed modes of formation of A) the aminocyclopentitol core unit of pactamycin and B) the mitosane core unit of mitomycin.

FIG. 13 is the LC-MS spectra of pactamycin, pactamycate, de-6MSA-pactamycin, and de-6MSA-pactamycate.

FIG. 14 is a series of graphs of response versus drug concentration illustrating the antimalarial activity of two pactamycin analogs (de-6MSA-pactamycin and de-6MSA-pactamycate) against chloroquine-sensitive (D6) and chloroquine-resistant (Dd2) strains of Plasmodium falciparum.

FIGS. 15A-15D are digital images of an agar diffusion activity of two pactamycin analogs (de-6MSA-pactamycin and de-6MSA-pactamycate against S. aureus (FIG. 15A), B. subtilis (FIG. 15B), E. coli (FIG. 15C), and P. aeruginosa (FIG. 15D). 1—pactamycin, 2—pactamycate, 3—de-6MSA-pactamycin, 4—de-6MSA-pactamycate.

FIGS. 16A-16D are dose response curves illustrating the dose-response of pactamycin and de-6MSA-pactamycin against S. aureus (FIG. 16A), M. smegmatis (FIG. 16B), P. aeruginosa (FIG. 16C), and E. coli (FIG. 16D).

FIGS. 17A-17B are graphs of cytotoxicity assays of two pactamycin analogs (de-6MSA-pactamycin and de-6MSA-pactamycate) against HT-29 cells in a sulforhodamine (SRB) assay (17A) and an MTT assay (17B).

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of the nucleic acid sequence is shown, but the complementary strand is understood as included by any reference to the displayed strand. In the accompanying sequence listing:

SEQ ID NOs: 1-46 are oligonucleotide sequences employed to amplify various genes within the pactamycin gene cluster.

SEQ ID NOs: 47 and 48 are oligonucleotide sequences employed to amplify the apramycin resistance gene.

SEQ ID NOs: 49 and 50 are oligonucleotide sequences employed to amplify the aminotransferase gene CetM.

SEQ ID NOs: 51-72 are oligonucleotide sequences employed to amplify various genes within the pactamycin gene cluster.

SEQ ID NOs: 73 and 74 are oligonucleotide sequences employed to amplify the ketosynthase domain in the rifB gene.

### DETAILED DESCRIPTION

### I. Overview of Several Embodiments

Pactamycin is isolated from the soil bacterium Streptomyces pactum (Bhuyan, Appl. Microbiol., 1962, 10:302-304). It shows potent antimicrobial (Id.), antitumor (White, Cancer Chemother. Rep., 1962, 24:75-78), antiviral (Taber et al., J. Virol., 1971, 8:395-401), and anti-protozoal (Otoguro et al., J. Antibiot., 2010, 63:381-384) activities, and literally affects cell growth of all three phylogenetic domains, eukarya, bacteria, and archaea. Its broad-spectrum growth inhibitory activity is mainly due to its ability to bind a conserved region within the 30S ribosomal subunit of most organisms, inhibiting the translocation of certain mRNA-tRNA complexes and blocking protein synthesis (Brodersen et al., Cell, 2000, 103:1143-1154; Dinos et al., Mol. Cell., 2004, 13:113-124). Although pactamycin was first reported in the 1960s and its various biological activities have been extensively investigated, further development of this compound has been hampered by its wide-ranging cytotoxicity. Moreover, its complex chemical structure has rendered its structural modification via synthetic organic chemistry difficult.

Disclosed herein are pactamycin analogs and methods for using the analogs.

In some embodiments, a disclosed compound is a compound other than pactamycin, 7-deoxypactamycin, or pactamycate having a chemical structure according to general formula (I)

where R¹ is H, lower aliphatic, amide, acyl, or aminoacyl; R² is —C(O)NR⁸R⁹ where R⁸ and R⁹ independently are hydrogen or lower aliphatic, or R¹ and R² together form a cyclic structure; R³ and R⁴ independently are hydrogen, hydroxyl, or lower aliphatic, or R² and R³ together form a cyclic structure; R⁵ is hydrogen or acyl; and R⁶ and R⁷ independently are hydrogen, hydroxyl, halogen, lower aliphatic, or amino.

In some embodiments, R⁶ is hydrogen, hydroxyl, lower aliphatic, or amino. In some embodiments, R⁷ is hydrogen, halogen, lower aliphatic, or amino.

In some embodiments, if R³, R⁸, and R⁹ are methyl, then at least one of R¹ or R⁴ is lower aliphatic or R⁶ is hydroxyl, lower aliphatic, or amino or R⁷ is halogen, lower aliphatic, or amino.

In some embodiments, if R³ is alkyl or hydrogen and R⁴ is hydrogen or hydroxyl, then at least one of R¹ or R⁵ is not hydrogen or R⁶ is hydroxyl, lower aliphatic, or amino or R⁷ is halogen, lower aliphatic, or amino.

In some embodiments, if R² and R³ together form a cyclic structure then R⁴ is not methyl or R¹ is not hydrogen or R⁶ is hydroxyl, lower aliphatic, or amino or R⁷ is halogen, lower aliphatic, or amino.

In any of all of the above embodiments, R³ and R⁴ may be hydrogen. In any or all of the above embodiments, R⁵ may be —C(O)R¹⁰ wherein R¹⁰ is

wherein X is selected from H, lower alkyl, —OR¹¹, halogen, —NO₂, —NR¹²R¹³; Y is H, halogen or lower alkyl; and R¹¹, R¹² and R¹³ independently are selected from H, lower alkyl and acyl.

In any or all of the above embodiments, R¹ and R² together may form a heterocyclic 5-membered ring, such as an imidazolidin-2-one ring. In any or all of the above embodiments, R² and R³ together may form a heterocyclic 5-membered ring, such as a 1,3-oxazolidin-2-one ring.

In any or all of the above embodiments, R⁵ may be a substituted benzoyl group, such as 2-hydroxy-6-methylbenzoyl.

In certain embodiments, the compound has a formula selected from

Embodiments of a pharmaceutical composition include a compound according to any or all or a subcombination thereof of the above embodiments, and a pharmaceutically acceptable carrier.

Embodiments of a method of inhibiting a tumor in a subject include selecting a subject for treatment that has, or is at risk for developing, a tumor, and administering to the subject an effective amount of a pharmaceutical composition according to any or all or a subcombination thereof of the above embodiments, thereby treating the tumor in the subject.

Embodiments of method of treating an infection from a pathogen of interest in a subject include selecting a subject for treatment that has, or is at risk for developing, an infection by a pathogen of interest, and administering to the subject a therapeutically effective amount of a pharmaceutical composition according to any or all of the above embodiments, thereby treating the infection form the pathogen of interest in the subject. In any or all of the above embodiments, the pathogen of interest may be a Gram-positive or Gram-negative bacterial pathogen.

Embodiments of a method of inhibiting growth of a pathogen include contacting the pathogen with a composition according to any or all of the above embodiments, wherein the composition is provided in an amount effective to inhibit the growth of the pathogen. In any or all of the above embodiments, the pathogen of interest may be a Gram-positive or Gram-negative bacterial pathogen.

Embodiments of a method of treating a parasitic infection in a subject include selecting a subject for treatment that has, or is at risk for developing, a parasitic infection, and administering to the subject an effective amount of a pharmaceutical composition comprising a compound according to any or all of the above embodiments. In any or all of the above embodiments, the parasitic infection may be malaria, Leishmaniasis, or Chagas Disease. In any or all of the above embodiments, the parasitic infection may result from Plasmodium falciparum. In certain embodiments, the compound has a formula selected from

### II. Terms

Unless otherwise noted, technical terms are used according to conventional usage. Definitions of common terms in molecular biology may be found in Benjamin Lewin Genes V published by Oxford University Press, 1994 (ISBN 0-19-854287-9); Kendrew et al. (eds.) The Encyclopedia of Molecular Biology, published by Blackwell Science Ltd., 1994 (ISBN 0-632-02182-9); and Robert A. Meyers (ed.) Molecular Biology and Biotechnology: a Comprehensive Desk Reference, published by VCH Publishers, Inc., 1995 (ISBN 1-56081-569-8).

To facilitate review of the various embodiments of this disclosure, the following explanations of specific terms are provided:

Acyl: A group of the formula RC(O)— wherein R is an organic group, such as an aliphatic or aromatic group.

Administration: To provide or give an agent to a subject, such as a therapeutic agent, by any effective route. Exemplary routes of administration include, but are not limited to, injection (such as subcutaneous, intramuscular, intradermal, intraperitoneal, intratumoral and intravenous), oral, intraductal, sublingual, rectal, transdermal, intranasal, and inhalation routes.

Aliphatic: Moieties including alkyl, alkenyl, alkynyl, halogenated alkyl and cycloalkyl groups as described below. A “lower aliphatic” group is a branched or unbranched aliphatic group having from 1 to 10 carbon atoms.

Alkyl: A branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 10 carbon atoms. The terms “halogenated alkyl” or “haloalkyl group” refer to an alkyl group as defined above with one or more hydrogen atoms present on these groups substituted with a halogen (F, Cl, Br, I). The term “cycloalkyl” refers to a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulfur, or phosphorous. Optionally substituted groups, such as “substituted alkyl,” describes groups, such as an alkyl group, having from 1-5 substituents, typically from 1-3 substituents, selected from alkoxy, optionally substituted alkoxy, acyl, acylamino, acyloxy, amino, aminoacyl, aminoacyloxy, aryl, carboxyalkyl, optionally substituted cycloalkyl, optionally substituted cycloalkenyl, optionally substituted heteroaryl, optionally substituted heterocyclyl, hydroxy, thiol and thioalkoxy.

Alkoxy: A straight, branched or cyclic hydrocarbon configuration and combinations thereof, including from 1 to 20 carbon atoms, preferably from 1 to 8 carbon atoms, more preferably from 1 to 4 carbon atoms, that include an oxygen atom at the point of attachment. An example of an “alkoxy group” is represented by the formula —OR, where R can be an alkyl group, optionally substituted with an alkenyl, alkynyl, aryl, aralkyl, cycloalkyl, halogenated alkyl, or heterocycloalkyl group as described above. Suitable alkoxy groups include methoxy, ethoxy, n-propoxy, i-propoxy, n-butoxy, i-butoxy, sec-butoxy, tert-butoxy cyclopropoxy, cyclohexyloxy, and the like.

Alkoxycarbonyl: An alkoxy substituted carbonyl, —C(O)OR, wherein R represents an optionally substituted alkyl, aryl, aralkyl, cycloalkyl, cycloalkylalkyl or similar moiety.

Alkenyl: A hydrocarbon group of 2 to 24 carbon atoms and structural formula containing at least one carbon-carbon double bond.

Aminoacyl: A group having the general formula

where R is an organic group, such as an aliphatic or aromatic group.

Amino: A chemical functional group —N(R)R′ where R and R′ are independently hydrogen, alkyl, heteroalkyl, haloalkyl, aliphatic, heteroaliphatic, aryl (such as optionally substituted phenyl or benzyl), heteroaryl, alkylsulfano, or other functionality. A “primary amino” group is —NH₂. “Mono-substituted amino” means a radical —N(H)R substituted as above and includes, e.g., methylamino, (1-methylethyl)amino, phenylamino, and the like. “Di-substituted amino” means a radical —N(R)R′ substituted as above and includes, e.g., dimethylamino, methylethylamino, di(1-methylethyl)amino, and the like.

Analog or mimetic: An analog is a molecule that differs in chemical structure from a parent compound, for example a homolog (differing by an increment in the chemical structure, such as a difference in the length of an alkyl chain), a molecular fragment, a structure that differs by one or more functional groups, a change in ionization. Structural analogs are often found using quantitative structure activity relationships (QSAR), with techniques such as those disclosed in Remington (The Science and Practice of Pharmacology, 19th Edition (1995), chapter 28). A mimetic is a molecule that mimics the activity of another molecule, such as a biologically active molecule. Biologically active molecules can include chemical structures that mimic the biological activities of a compound.

Animal: A living multi-cellular vertebrate or invertebrate organism, a category that includes, for example, mammals and birds. The term mammal includes both human and non-human mammals. Similarly, the term “subject” includes both human and veterinary subjects, such as non-human primates. Thus, administration to a subject can include administration to a human subject. Particular examples of veterinary subjects include domesticated animals (such as cats and dogs), livestock (for example, cattle, horses, pigs, sheep, and goats), laboratory animals (for example, mice, rabbits, rats, gerbils, guinea pigs, and non-human primates), as well as birds, reptiles, fish, and mollusks.

Antibiotic: A compound, composition, or substance that inhibits the growth and/or kills bacteria. The term antibiotic can also be used to refer to more than one antibiotic. Examples of antibiotics include without limitation, aminoglycosides (such as amikacin, gentamicin, kanamycin, neomycin, netilmicin, streptomycin, tobramycin, and paromomycin); ansamycins (such as geldanamycin, and herbimycin); carbacephems (such as loracarbef, ertapenem, doripenem, imipenem/cilastatin, and meropenem); cephalosporins (such as cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, and ceftobiprole); glycopeptides (such as teicoplanin and vancomycin); macrolides (such as azithromycin, clarithromycin, dirithromycin, erythromycin, roxithromycin, troleandomycin, telithromycin, and spectinomycin); monobactams (such as aztreonam); penicillins (such as amoxicillin, ampicillin, azlocillin, carbenicillin, cloxacillin, dicloxacillin, flucloxacillin, mezlocillin, meticillin, nafcillin, oxacillin, penicillin, piperacillin, and ticarcillin); polypeptides (such as bacitracin, colistin, and polymyxin b); quinolones (such as ciprofloxacin, enoxacin, gatifloxacin, levofloxacin, lomefloxacin, moxifloxacin, norfloxacin, ofloxacin, trovafloxacin, grepafloxacin, and sparfloxacin); sulfonamides (such as mafenide, prontosil (archaic), sulfacetamide, sulfamethizole, sulfanilamide (archaic), sulfasalazine, sulfisoxazole, trimethoprim, and trimethoprim-sulfamethoxazole); tetracyclines (such as demeclocycline, doxycycline, minocycline, oxytetracycline, and tetracycline); and others (such as arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampicin, thiamphenicol, and tinidazole). Antibiotics are often produced by or derived from certain fungi, bacteria, and other organisms. In some examples, disclosed pactamycin analogs or combinations thereof are antibiotics.

Bacterial pathogen: A bacteria that causes disease (pathogenic bacteria). Examples of pathogenic bacteria include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginale, Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Plesiomonas shigelloides. Prevotella sp., Porphyromonas sp., Prevotella melaminogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia among others.

Carbamate: A group of the formula —OC(O)NH—.

Carbonyl: A —C(O)— group. Carbonyl-containing groups include any substituent containing a carbon-oxygen double bond (C═O), including acyl groups, amides, carboxy groups, esters, ureas, carbamates, carbonates and ketones and aldehydes, such as substituents based on —COR or —RCHO where R is an aliphatic, heteroaliphatic, alkyl, heteroalkyl, hydroxyl, or a secondary, tertiary, or quaternary amine.

Carboxyl: A —COOH group. Substituted carboxyl refers to —COOR where R is aliphatic, heteroaliphatic, alkyl, heteroalkyl, or a carboxylic acid or ester.

Cell: A plant, animal, insect, bacterial, or fungal cell.

Chagas Disease: A tropical parasitic disease caused by the flagellate protozoan Trypanosoma cruzi. T. cruzi is commonly transmitted to humans and other mammals by an insect vector, such as the insects of the subfamily Triatominae (family Reduviidae), most commonly species belonging to the Triatoma, Rhodnius, and Panstrongylus genera. The disease may also be spread through blood transfusion and organ transplantation, ingestion of food contaminated with parasites, and from a mother to her fetus.

Culturing: The process by which either prokaryotic or eukaryotic cells are grown under controlled conditions. Culture conditions vary widely for each cell type, and variation of conditions for a particular cell type can result in different phenotypes being expressed. Aside from temperature and gas mixture, the most commonly varied factor in culture systems is the growth medium. Recipes for growth media can vary in pH, glucose concentration, growth factors, and the presence of other nutrient components. As used herein, culturing refers to the growing of cells, such as S. lividans cells under conditions that allow the production of 7-demethyl-7-deoxypactamycin and de-6MSA-7-demethyl-7-deoxypactamycin and variants thereof.

Derivative: A compound or portion of a compound that is derived from or is theoretically derivable from a parent compound.

Effective amount: A amount or quantity of a specific substance (for example 7-demethyl-7-deoxypactamycin, de-6MSA-7-demethyl-7-deoxypactamycin or a combination thereof) sufficient to achieve a desired response, such as reducing or inhibiting one or more signs or symptoms associated with a condition or disease. For instance, this can be the amount necessary to inhibit or treat an infection by a pathogen, such as an infection by a bacterial or protozoan pathogen. When administered to a subject, a dosage will generally be used that will achieve a desired in vivo effect. In some examples, an “effective amount” is one that treats one or more symptoms and/or underlying causes of any of a disorder or disease. In some examples, an “effective amount” is a therapeutically effective amount in which the agent alone or with an additional therapeutic agent(s) (for example a chemotherapeutic agent), induces the desired response such as treatment of a tumor. In one example, a desired response is to decrease tumor size or metastasis in a subject to whom the therapy is administered. Tumor growth or metastasis does not need to be completely eliminated for the composition to be effective. For example, an agent can decrease tumor growth by a desired amount, for example by at least 20%, at least 50%, at least 60%, at least 70%, at least 80%, at least 90%, at least 95%, at least 98%, or even at least 100% (elimination of the tumor growth), as compared to growth in the absence of the composition.

A therapeutically effective amount of a substance can be administered in a single dose, or in several doses, for example daily, during a course of treatment. However, the effective amount of a composition will be dependent on the compound applied, the subject being treated, the severity and type of the affliction, and the manner of administration of the composition. For example, a therapeutically effective amount of composition can vary from about 0.01 mg/kg body weight to about 1 g/kg body weight.

Fungal pathogen: A fungus that causes disease. Examples of fungal pathogens include without limitation Trichophyton rubrum, T. mentagrophytes, Epidermophyton floccosum, Microsporum canis, Pityrosporum orbiculare (Malassezia furfur), Candida sp. (such as Candida albicans), Aspergillus sp. (such as Aspergillus fumigatus, Aspergillus flavus and Aspergillus clavatus), Cryptococcus sp. (such as Cryptococcus neoformans, Cryptococcus gattii, Cryptococcus laurentii and Cryptococcus albidus), Histoplasma sp. (such as Histoplasma capsulatum), Pneumocystis sp. (such as Pneumocystis jirovecii), and Stachybotrys (such as Stachybotrys chartarum) among others.

Hydroxyl: —OH. The term “hydroxyalkyl” refers to an alkyl group that has at least one hydrogen atom substituted with a hydroxyl group. The term “alkoxyalkyl group” is defined as an alkyl group that has at least one hydrogen atom substituted with an alkoxy group described above.

Inhibit: To decrease, reduce, limit or block the action or function of a molecule. In an example, to inhibit includes to inhibit or reduce tumor growth by at least 10%, at least 20%, at least 50%, at least 70%, or even at least 90%, such as about a 10% to 90% reduction, about a 20% to 70% reduction, about a 30% to 60% reduction, about a 40% to 50% reduction, including a 15%, 20%, 25%, 30%, 35%, 40%, 45%, 50%, 60%, 65%, 70%, 75%, 85%, 90%, 95% or greater decrease in tumor size, proliferation, or combination thereof. In some examples, inhibit includes inhibiting the full development of a disease or condition, for example, in a subject who is at risk for a disease such as an infection with a pathogen, for example a bacterial pathogen.

Isolated: An isolated biological component (such as a nucleic acid molecule or protein or antimicrobial agent, such as a disclosed pactamycin analog) is one that has been substantially separated or purified away from other biological components in the cell of the organism in which the component naturally occurs, such as other chromosomal and extra-chromosomal DNA and RNA, proteins and organelles. With respect to nucleic acids and/or polypeptides, the term can refer to nucleic acids or polypeptides that are no longer flanked by the sequences typically flanking them in nature. Nucleic acids and proteins that have been isolated include nucleic acids and proteins purified by standard purification methods. The term also embraces nucleic acids and proteins prepared by recombinant expression in a host cell as well as chemically synthesized nucleic acids. The term also embraces antimicrobial agents, such as thereof, prepared by recombinant expression in a host cell as well as chemically synthesized antimicrobial agents, such as a disclosed pactamycin analog (e.g., 7-demethyl-7-deoxypactamycin and/or de-6MSA-7-demethyl-7-deoxypactamycin).

Leishmaniasis: A disease caused by protozoan parasites that belong to the genus Leishmania and is transmitted by the bite of certain species of sand fly (subfamily Phlebotominae). Most forms of the disease are transmissible only from animals, but some can be spread between humans. Human infection is caused by about 21 species that infect mammals, including the L. donovani complex (including L. donovani, L. infantum, and L. chagasi); the L. mexicana complex (including L. mexicana, L. amazonensis, and L. venezuelensis); L. tropica; L. major; L. aethiopica; and the subgenus Viannia with four main species (L. (V.) braziliensis, L. (V.) guyanensis, L. (V.) panamensis, and L. (V.) peruviana).

Malaria: A tropical disease, spread by mosquitoes from person to person, that exacts a devastating toll in endemic regions, especially Africa, where it claims 1 to 2 million lives each year. The deaths occur primarily among young children and pregnant women—vulnerable populations for whom therapeutic options are limited. These options are even more restricted in the current landscape of widespread drug resistance in the Plasmodium parasites (protozoans) that cause malaria. Together with an increasing incidence of malaria worldwide, there is an urgent and unmet need for new drugs to prevent and treat malaria, an infection that causes clinical disease manifestations in 300 to 500 million people each year.

Multidrug-resistant or drug-resistant: A term that (when used herein) refers to malaria, or the parasites causing malaria, that have developed resistance to treatment by at least one therapeutic agent historically administered to treat malaria. For example, there are multidrug-resistant strains of Plasmodium falciparum that harbor high-level resistance to chloroquine, quinine, mefloquine, pyrimethamine, sulfadoxine and atovaquone.

Open reading frame (ORF): A series of nucleotide triplets (codons) coding for amino acids without any internal termination codons. These sequences are usually translatable into a peptide. For example, ORF, open reading frame, and pactamycin ORF refer to an open reading frame in the pactamycin biosynthetic gene cluster as isolated from Streptomyces pactum. The term also embraces the same ORFs as present in other pactamycin-synthesizing organisms. The term encompasses allelic variants and single nucleotide polymorphisms (SNPs). In certain instances the term pactamycin ORF is used synonymously with the polypeptide encoded by the pactamycin ORF and may include conservative substitutions in that polypeptide. The particular usage will be clear from context.

Pactamycin: A structurally unique aminocyclitol antibiotic isolated from Streptomyces pactum, consists of a 5-member ring aminocyclitol (cyclopentitol) unit, two aromatic rings (6-methyl salicylic acid 3-aminoacetophenone) and a 1,1-dimethylurea (Wiley et al., J. Org. Chem., 35: 1420-1425, 1970; and Rinehart et al., J. Nat. Prod. 43: 1-20, 1979). It has been suggested that the five-member ring aminocyclitol moiety of pactamycin is derived from glucose, whereas the 6-methyl salicylic acid is derived from acetic acid. The 3-aminoacetophenone moiety is derived from an unknown branch of the amino-shikimate pathway. The four-methyl groups and the hydroxymethine carbon in the molecule are derived from methionine (Weller and Rinehart, J. Am. Chem. Soc., 100: 6757-6760, 1978). Pactamycin has potent antibacterial activities against Gram-positive and Gram-negative bacteria (Bhuyan, Appl. Microbiol., 10: 302-304, 1962). It also shows a strong anti-tumor activity.

Pathogen: A disease-producing agent. Examples include, but are not limited to, viruses, bacteria, protozoa, parasites, and fungi.

Pharmaceutically acceptable salt or pharmacologically acceptable salt: Salts prepared by conventional means that include basic salts of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. “Pharmaceutically acceptable salts” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977).

Pharmaceutically acceptable carriers: The pharmaceutically acceptable carriers of use with the disclosed methods are conventional carriers. Remington's Pharmaceutical Sciences, by E. W. Martin, Mack Publishing Co., Easton, Pa., 19th Edition, 1995, describes compositions and formulations suitable for pharmaceutical delivery of pactamycin analogs, such as 7-demethyl-7-deoxypactamycin and de-6MSA-7-demethyl-7-deoxypactamycin.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent or drug: A chemical compound or composition capable of inducing a desired therapeutic or prophylactic effect when properly administered to a subject.

Purified or substantially purified: The term purified does not require absolute purity; rather, it is intended as a relative term. Thus, for example, a compound is one in which the compound referred to is more pure than the other compounds or contaminants within the mixture. Substantially pure or substantially purified refers to a condition in which the mixture contains at least 80% of the compound referred to such as pactamycin or an analog thereof, such as at least 85%, at least 90%, at least 95%, at least 98%, including about 80% to 95%, about 90% to 98%, about 95% to 99%, such as 81%, 82%, 83%, 84%, 85%, 86%, 87%, 88%, 89%, 90%, 91%, 92%, 93%, 94%, 95%, 96%, 97%, 98%, 99% or even 100% pure.

Treating a disease: A phrase referring to a therapeutic intervention that ameliorates a sign or symptom of a disease or pathological condition after it has begun to develop. The term “ameliorate” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology. A “prophylactic” treatment can also include a treatment administered to a subject to prevent or reduce the reoccurrence of a sign or symptom of a particular disease, such as reoccurrence of tumor growth.

Tumor, neoplasia, malignancy or cancer: The result of abnormal and uncontrolled growth of cells. Neoplasia, malignancy, cancer and tumor are often used interchangeably and refer to abnormal growth of a tissue or cells that results from excessive cell division. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” “Malignant cells” are those that have the properties of anaplasia invasion and metastasis.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” In case of conflict, the present specification, including explanations of terms, will control. In addition, the materials, methods, and examples are illustrative only and not intended to be limiting.

Suitable methods and materials for the practice of the disclosed embodiments are described below. In addition, any appropriate method or technique well known to the ordinarily skilled artisan can be used in the performance of the disclosed embodiments. Some conventional methods and techniques applicable to the present disclosure are described, for example, in Sambrook et al., Molecular Cloning: A Laboratory Manual, 2^{nd} ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999.

All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Description of Representative Embodiments

A. Pactamycin Analogs

Pactamycin is a complex and densely functionalized aminocyclitol antibiotic. The unusual core aminocyclopentitol structure of pactamycin offers great potential as a scaffold for generating new biologically active compounds.

However, efforts to modify the chemical structure of pactamycin using conventional organic synthesis methodology have been difficult due to the complexity of the molecule, calling for an alternative approach to generate structural modifications, e.g., via biosynthetic approaches. The biosynthetic gene cluster of pactamycin has been identified within an 86 kb sequenced region of DNA from S. pactum ATCC 27456. Functional analysis of the genes has been performed by a combination of bioinformatics, enzymatic assays, gene inactivation, as well as chemical and genetic complementation studies. These studies are described in U.S. patent application Ser. No. 12/596,429 filed on Oct. 16, 2009 (and published as US 2010/0210837 A1), which is the U.S. National Stage of International Application No. PCT/US2008/060876, filed Apr. 18, 2008 (and published as WO 2008/131258), which in turn claims the benefit of the earlier filing date of U.S. Provisional Application No. 60/912,824, filed Apr. 19, 2007; each of these applications is hereby incorporated by reference in its entirety.

Disclosed herein are pactamycin analogs. The disclosed analogs have antimicrobial activities as well as other useful pharmaceutical activities including antitumor activities. In some examples, the analogs include at least one of antibacterial, antitumor, or antimalarial activity. It is contemplated that additional inhibitory activities can be present including antifungal or other antiparasitic activities. In some embodiments, the antibacterial activities include inhibitory activity of Gram-positive bacteria (such as Bacillus subtilis) and/or Gram-negative bacteria (such as Escherichia coli). In some embodiments, the disclosed analogs have inhibitory activity against one or more strains of Plasmodium falciparum. In some embodiments, a disclosed analog has inhibitory activity against a chloroquine-sensitive strain of Plasmodium falciparum. In some embodiments, a disclosed analog has inhibitory activity against a chloroquine-sensitive strain of Plasmodium falciparum.

Some embodiments of the disclosed pactamycin analogs have a chemical structure according to general formula (I)

where R¹ is H, lower aliphatic, amide, acyl, or aminoacyl; R² is —C(O)NR⁸R⁹ where R⁸ and R⁹ independently are hydrogen or lower aliphatic, or R¹ and R² together form a cyclic structure; R³ and R⁴ independently are hydrogen, hydroxyl, or lower aliphatic, or R² and R³ together form a cyclic structure; R⁵ is hydrogen or acyl; R⁶ and R⁷ independently are hydrogen, hydroxyl, halogen, lower aliphatic, or amino.

In some embodiments, R⁵ is an acyl group represented by —C(O)R¹⁰ wherein R¹⁰ is an aromatic group, such as an optionally substituted mono- or polycyclic aromatic group. By way of example, such R¹⁰ groups include, without limitation:

wherein X is selected from H, lower alkyl, —OR¹¹, halogen, —NO₂, —NR¹²R¹³; Y is H, halogen or lower alkyl; and R¹¹, R¹² and R¹³ independently are selected from H, lower alkyl and acyl.

In some embodiments, R⁶ is hydrogen, hydroxyl, lower aliphatic, or amino. In some embodiments, R⁷ is hydrogen, halogen, lower aliphatic, or amino.

In one embodiment, if R³, R⁸, and R⁹ are methyl, then at least one of R¹ or R⁴ is lower aliphatic or R⁶ is hydroxyl, lower aliphatic, or amino or R⁷ is halogen, lower aliphatic, or amino. In one embodiment, if R³ is alkyl or hydrogen and R⁴ is hydrogen or hydroxyl, then at least one of R¹ or R⁵ is not hydrogen or R⁶ is hydroxyl, lower aliphatic, or amino or R⁷ is halogen, lower aliphatic, or amino.

In some embodiments, R¹ and R² together or R² and R³ together form a 5-membered ring, such as a heterocyclic 5-membered ring. In one embodiment, R¹ and R² together form a carbonyl-containing, heterocyclic 5-membered ring, such as an imidazolidin-2-one ring. In one embodiment, R² and R³ together form a carbamate-containing, heterocyclic 5-membered ring, such as a 1,3-oxazolidin-2-one ring. In one embodiment, if R² and R³ together form a cyclic structure, then R⁴ is not methyl or R¹ is not hydrogen or R⁶ is hydroxyl, lower aliphatic, or amino or R⁷ is halogen, lower aliphatic, or amino.

In some embodiments, R⁵ is derived from substituted or unsubstituted pyrrole-2-carboxylic acids, furoic acids, benzoic acids, benzothiphene-2-carboxylic acids, or thiazole-carboxylic acids. In certain embodiments, R⁵ is a substituted benzoate, such as 2-hydroxy-6-methylbenzoate (6-methyl salicylic acid (6MSA)).

In particular embodiments, R³ and R⁴ are hydrogen, and the compound has a chemical structure according to general formula (II)

where R¹, R², and R⁵ are as defined above.

Exemplary pactamycin analogs are shown in Table 1.

```
TABLE 1
  7-demethyl-7-deoxypactamycin
  N-demethylpactamycin
  N,N-didemethylpactamycin
  N-demethyl-7-deoxypactamycin
  N,N-didemethyl-7-deoxypactamycin
  8″-hydroxypactamycin
  8″-hydroxy-7-deoxypactamycin
  5″-fluoropactamycin
  5″-fluoropactamycate
  8″-hydroxypactamycate
  pactalactam
  7-deoxypactalactam
  7-demethyl-7-deoxypactalactam
  8″-hydroxy-7-deoxypactalactam
  de-6MSA-pactamycin
  de-6MSA-7-demethyl-7- deoxypactamycin
  de-6MSA-7-demethyl-7- deoxypactalactam
  de-6MSA-pactamycate
```

B. Pharmaceutical Compositions

Also disclosed herein are pharmaceutical compositions including a pharmacologically active amount of at least one disclosed pactamycin analog or a pharmaceutically acceptable salt thereof, and at least one pharmaceutically acceptable carrier. The disclosed pharmaceutical compositions are formulated for use in human or veterinary medicine. Embodiments of pharmaceutical compositions include a pharmaceutically acceptable carrier and at least one active ingredient. Useful pharmaceutically acceptable carriers and excipients are known in the art. In some examples, active ingredients include at least one pactamycin compound such as a pactamycin analog as described herein. In addition, other medicinal or pharmaceutical agents, for example, with similar, related or complementary effects on the affliction being treated, may be included as active ingredients in pharmaceutical compositions.

The pharmaceutical compositions including pactamycin-like antibiotics may be formulated in a variety of ways depending, for example, on the mode of administration and/or on the location and type of disease to be treated. For example, such pharmaceutical compositions may be formulated as pharmaceutically acceptable salts. As another example, parenteral formulations may comprise injectable fluids that are pharmaceutically and physiologically acceptable fluid vehicles such as water, physiological saline, other balanced salt solutions, aqueous dextrose, glycerol or the like. Excipients may include, for example, nonionic solubilizers, such as cremophor, or proteins, such as human serum albumin or plasma preparations. If desired, the pharmaceutical composition to be administered may also contain non-toxic auxiliary substances, such as wetting or emulsifying agents, preservatives, and pH buffering agents and the like, for example, sodium acetate or sorbitan monolaurate.

The dosage form of the pharmaceutical composition will be determined by the mode of administration chosen. For example, in addition to injectable fluids, topical and oral formulations may be employed. Topical preparations may include eye drops, ointments, sprays and the like. Oral formulations may be liquid (e.g., syrups, solutions or suspensions), or solid (e.g., powders, pills, tablets, or capsules). For solid compositions, conventional non-toxic solid carriers may include pharmaceutical grades of mannitol, lactose, starch, or magnesium stearate. Actual methods of preparing such dosage forms are known, or will be apparent, to those skilled in the art.

Certain embodiments of the pharmaceutical compositions including one or more of the disclosed pactamycin analogs as described herein may be formulated in unit dosage form suitable for individual administration of precise dosages. The amount of a therapeutic compound administered will depend on the subject being treated, the severity of the affliction, and the manner of administration, and is known to those skilled in the art. Within these bounds, the formulation to be administered will contain a quantity of the pactamycin analogs disclosed herein in an amount effective to achieve the desired effect in the subject being treated (e.g., eliminating Gram-positive pathogens, Gram-negative pathogens, malaria or providing anti-tumor activity).

C. Methods of Producing and Isolating Pactamycin Analogs

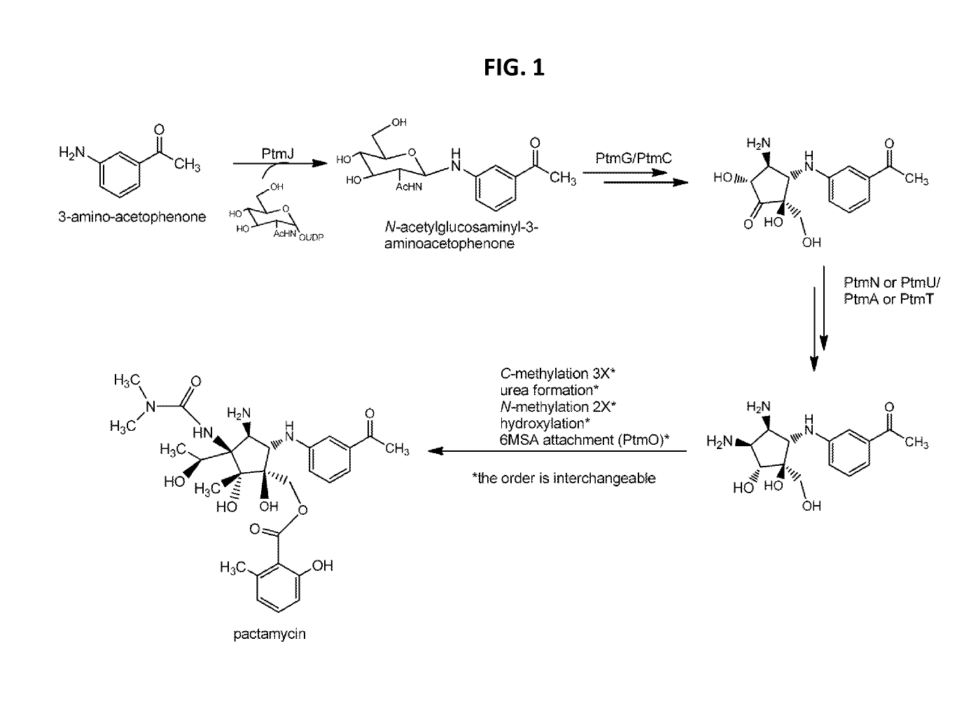

Methods of producing and isolating pactamycin analogs are also disclosed. A proposed biosynthetic pathway of pactamycin is illustrated in FIG. 1. In some embodiments, a method of synthesizing a pactamycin analog includes inactivation of one or more genes in the S. pactum pactamycin gene cluster, such as the ptmH gene or ptmD gene in S. pactum, to produce pactamycin analogs by altering various functional groups on pactamycin. For example, 7-demethyl-7-deoxypactamycin is synthesized by inactivation of the ptmH gene in S. pactum. Further, de-6MSA-7-demethyl-7-deoxypactamycin and N,N-didemethyl-pactamycin are synthesized by inactivation of the ptmD gene in S. pactum. Inactivation of the ptmQ gene in S. pactum produces de-6MSA-pactamycin. Inactivation of the ptmH and ptmQ genes in S. pactum produces de-6MSA-7-demethyl-7-deoxypactamycin. Additional methods of producing and isolating pactamycin analogs are disclosed in Examples 1 and 2 below and PCT Publication No. WO 2008/131258, which is incorporated in its entirety herein by reference.

D. Methods of Using Pactamycin Analogs

Methods of using pactamycin analogs (alone or in combination(s)) are provided herein. For example, methods of treating or preventing infection, such as a parasitic or bacterial infection, with one or more disclosed pactamycin analogs are provided. Also disclosed are methods of treating a tumor with one or more disclosed pactamycin analogs.

In some examples, methods of treating a subject for infection with pathogenic organisms are disclosed. For example, methods for inhibiting a pathogen of interest, including bacteria (e.g., Gram-positive bacteria or Gram-negative bacteria), a fungus, a virus or a parasite (such as Plasmodium falciparum) of interest are provided. In other examples, a method is provided for treating a subject with a parasitic disease, including but not limited to malaria (including drug-resistant malaria), Leishmaniasis, or Chagas disease, is provided. Such treatments include administering a pactamycin analog, or a combination of one or more pactamycin analogs and one or more other pharmaceutical agents (also referred to herein as “drug” or “drugs”), to the subject in a pharmaceutically acceptable carrier and in an amount effective to treat the pathogen of interest. By “treat,” “treating,” or “treatment” is meant a method of reducing the effects of an existing infection. Treatment can also refer to a method of reducing the disease or condition itself rather than just the symptoms. The treatment can be any reduction from native levels and can be, but is not limited to, the complete ablation of the disease or the symptoms of the disease. Treatment can range from a reduction in a symptom or symptoms of infection to complete amelioration of the infection as detected by art-known techniques. For example, a disclosed method is considered to be an effective treatment if there is about a 10% reduction, such as about 20%, 30%, 40%, 50%, 60%, 70%, 80%, 90%, or 100% in one or more symptoms of the disease in a subject with the disease when compared to native levels in the same subject or control subjects.

Methods for treating an existing pathogen infection in a subject, or for providing a prophylactic effect to a subject who is susceptible to a pathogen infection, are disclosed. For example, the disclosed methods can be used to prevent, such as preclude, delay, avert, obviate, forestall, stop, or hinder the onset, incidence, severity, or recurrence of infection. For example, the disclosed method is considered to be a prevention if there is about a 10% reduction in onset, incidence, severity, or recurrence of infection, or symptoms of infection (e.g., inflammation, fever, lesions, weight loss, etc.) in a subject exposed to an infection when compared to control subjects exposed to an infection that did not receive a composition for decreasing infection. Thus, the reduction in onset, incidence, severity, or recurrence of infection can be about a 10%, 20%, 30%, 40%, 50%, 60%, 70%, 80%, 90%, 100%, 150%, 200%, 500% or any amount of reduction as compared to control subjects. For example, and not to be limiting, if about 10% of the subjects in a population do not become infected as compared to subjects that did not receive preventive treatment, this is considered prevention.

Subjects can be selected using more specific criteria, such as a definitive diagnosis of a condition based on, for example, a biological specimen that has been provided to be tested for a bacterial or parasitic infection. In some examples, the treatment methods include screening a subject for an infection prior to administering a disclosed treatment. In particular examples, the subject is screened to determine if the pathogenic infection is a parasitic infection. Examples of methods that can be used to screening for a parasitic include a combination of a tissue biopsy, and serum blood levels. If blood or a fraction thereof (such as serum) is used, 1-100 μl of blood is collected. Serum can either be used directly or fractionated using filter cut-offs to remove high molecular weight proteins. If desired, the serum can be frozen and thawed before use. If a tissue biopsy sample is used, 1-100 μg of tissue is obtained, for example using a fine needle aspirate. The biological sample (e.g., tissue biopsy or serum) is analyzed for the presence of a pathogenic infection, such as a parasitic infection. If the infection is a parasitic infection, it can be treated with the disclosed therapies.

Identification of subjects with the same medical condition, such as a pathogenic infection, such as a parasitic infection, can be accomplished by selecting all patients with the same diagnosis within electronic health records (EHR). EHRs are individual health records in a digitized format that can be accessed via a computer or computer-based system over a network. EHRs are designed to keep information about each encounter with the patient. For example, EHRs may include a person's health characteristics, medical history, past and current diagnoses, lab reports and results, x-rays, photographs, prescribed medication, billing and insurance information, contact information, demographics, and the like.

In other examples, the present disclosure contemplates treatments for tumors, such as cancer including colorectal cancer, head and neck squamous cell carcinomas, and other skin cancers. In some examples, such treatments include administering an effective amount of a pactamycin analog, or a combination of one or more disclosed pactamycin analogs and one or more other pharmaceutical agents, to the subject in a pharmaceutically acceptable carrier and in an amount effective to reduce or eliminate the tumor, such as inhibiting tumor growth, relative to a control. The control can be any suitable control, such as a reference value. For example, the reference value can be an historical value based on average activity or expression of a protein or mRNA known to be present in a subject with a tumor, such as cancer. In some examples, a control is biological sample obtained from the subject with a tumor prior to administration of a disclosed analog. A disclosed method is considered to be an effective treatment if there is about a 10% reduction, such as about 20%, 30%, 40%, 50%, 60%, 70%, 80%, 90%, or 100% in one or more signs or symptoms associated with the tumor in the subject when compared to native levels in the same subject or control subjects.

Subjects can be selected using more specific criteria, such as a definitive diagnosis of a condition based on, for example, a biological specimen that has been provided to be tested for tumor cells. In some examples, the treatment methods include screening a subject for a tumor prior to administering a disclosed treatment. In particular examples, the subject is screened to determine if the tumor is malignant, indicating cancer, or benign. Examples of methods that can be used to screening for a tumor include a combination of ultrasound, tissue biopsy, and serum blood levels. If blood or a fraction thereof (such as serum) is used, 1-100 μl of blood is collected. Serum can either be used directly or fractionated using filter cut-offs to remove high molecular weight proteins. If desired, the serum can be frozen and thawed before use. If a tissue biopsy sample is used, 1-100 μg of tissue is obtained, for example using a fine needle aspirate. The biological sample (e.g., tissue biopsy or serum) is analyzed for the presence of a malignant tumor, such as cancer. If the tumor is malignant, it can be treated with the disclosed therapies.

Identification of subjects with the same medical condition, such as a tumor, including cancer, can be accomplished by selecting all patients with the same diagnosis within electronic health records (EHR). EHRs are simply individual health records in a digitized format that can be accessed via a computer or computer-based system over a network. EHRs are designed to keep information about each encounter with the patient. For example, EHRs may include a person's health characteristics, medical history, past and current diagnoses, lab reports and results, x-rays, photographs, prescribed medication, billing and insurance information, contact information, demographics, and the like.

The vehicle in which the drug is delivered may include, for example, the pharmaceutical compositions described above. Routes of administration include but are not limited to oral and parenteral routes, such as intravenous (iv), intraperitoneal (ip), rectal, topical, ophthalmic, nasal, and transdermal.

Therapeutically effective doses of a pactamycin analog can be determined by one of skill in the art. An example of a dosage range is 0.1 to 200 mg/kg body weight orally in single or divided doses. Another example of a dosage range is 1.0 to 100 mg/kg body weight orally in single or divided doses. For oral administration, the compositions are, for example, provided in the form of a tablet containing 0.1 or 1.0 to 1000 mg of the active ingredient, particularly 1, 5, 10, 15, 20, 25, 50, 100, 200, 400, 500, 600, and 1000 mg of the active ingredient for the symptomatic adjustment of the dosage to the subject being treated.

The specific dose level and frequency of dosage for any particular subject may be varied and will depend upon a variety of factors, including the activity of the specific pactamycin compound, the metabolic stability and length of action of that compound, the age, body weight, general health, sex and diet of the subject, mode and time of administration, rate of excretion, drug combination, and severity of the condition of the subject undergoing therapy.

The disclosed methods for inhibiting or treating a disease, such as cancer (e.g., colorectal cancer), or an infection, such as a parasitic infection, can be used alone or can be accompanied by administration of other agents.

In some examples, methods for inhibiting tumor growth include administering an effective amount of a disclosed pactamycin analog and at least one anti-cancer agent or therapeutic treatment (such as surgical resection of a tumor or radiation therapy). Any suitable anti-cancer agent can be administered to a patient as part of a treatment regimen that includes inhibiting or treating a malignant tumor. Exemplary anti-cancer agents include, but are not limited to, chemotherapeutic agents, such as, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (e.g. anti-androgens) and anti-angiogenesis agents. Other anti-cancer treatments include radiation therapy and antibodies that specifically target cancer cells.

Examples of alkylating agents include nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine).

Examples of antimetabolites include folic acid analogs (such as methotrexate), pyrimidine analogs (such as 5-FU or cytarabine), and purine analogs, such as mercaptopurine or thioguanine.

Examples of natural products include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitomycin C), and enzymes (such as L-asparaginase).

Examples of miscellaneous agents include platinum coordination complexes (such as cis-diamine-dichloroplatinum II also known as cisplatin), substituted ureas (such as hydroxyurea), methyl hydrazine derivatives (such as procarbazine), and adrenocortical suppressants (such as mitotane and aminoglutethimide).

Examples of hormones and antagonists include adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testerone proprionate and fluoxymesterone).

Examples of many of the most commonly used chemotherapy drugs include Adriamycin, Alkeran, Ara-C, BiCNU, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, while some more newer drugs include Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol.

In some examples, methods for inhibiting or treating a parasitic infection include administering an effective amount of a disclosed pactamycin analog and at least one other compound selected from chloroquine, quinine, sulfadoxine, pyrimethamine, primaquine, artemisinin, mefloquine, atovaquone, proguanil, pentamidine, isethionate, melarsoprol, eflornithine, nifurtimox, and benznidazole.

When used in combination with the administration of one of the disclosed pactamycin analogs, the additional treatment methods described above can be administered or performed prior to, at the same time, or following the disclosed anti-tumor or anti-infection therapy as appropriate for the particular patient, the additional symptoms associated with the tumor or infection (e.g., hormonal symptoms, conditions and related diseases) and the specific combination of therapies.

The following examples are provided to illustrate certain particular features and/or embodiments. These examples should not be construed to limit the disclosure to the particular features or embodiments described.

### Example 1

### Production and Analysis of de-6MSA-7-demethyl-7-deoxypactamycin (TM-025) and 7-demethyl-7-deoxypactamycin (TM-026)

This example describes the production and analysis of de-6MSA-7-demethyl-7-deoxypactamycin (referred herein as TM-025) and 7-demethyl-7-deoxypactamycin (referred to herein as TM-026).

Bacterial Strains and Plasmids:

Pactamycin producing S. pactum ATCC 27456 was purchased from American Type Culture Collection (ATCC). Escherichia coli DH10B was used as a host strain for the construction of recombinant plasmids. E. coli ET12567 was used as donor strain in conjugation experiments. pBlueScript II (SK−) (Stratagene) and pGEM-T Easy (Promega) were used as cloning vectors. pJTU695 and pSET152 were used for construction of integrative shuttle plasmids for complementation studies and pOJ446 was used for amplification of Apr^{R} gene (Kieser et al., “Practical Streptomyces Genetics,” 2000, The John Innes Foundation, Norwich, England). pHZ1358 is a pIJ101 derivative containing an OriT transfer element required for conjugation (Sun et al., Microbiology, 2002, 148:361-371). Other bacterial strains and plasmids used in this study are listed in Table 2.

TABLE 2

| | Relevant genotype/comments | Source/Ref |
|---|---|---|
| Strains | | |
| Escherichia coli DH10B | F⁻ mcrA Δ(mrr-hsdRMS-mcrBC) φ80lacZΔM15 | GibcoBRL |
| | ΔlacX74 recA1 endA1 araD139 Δ(ara, | |
| | leu)7697 galU galK λ⁻ rspL nupG | |
| E. coli ET12567(pUZ8002) | dam dcm hsdS, pUZ8002 | (1) |
| Streptomyces pactum ATCC | Wild-type pactamycin producing strain | ATCC |
| 27456 | | |
| S. pactum ΔptmB | ptmB disruption mutant | Disclosed |
| | | herein |
| S. pactum ΔptmD | ptmD disruption mutant | Disclosed |
| | | herein |
| S. pactum ΔptmH | ptmH disruption mutant | Disclosed |
| | | herein |
| S. pactum ΔptmL | ptmL disruption mutant | Disclosed |
| | | herein |
| S. pactum ΔptmM | ptmM disruption mutant | Disclosed |
| | | herein |
| S. pactum ΔptmY | ptmY disruption mutant | Disclosed |
| | | herein |
| S. pactum ΔptmH-ptmQ | ptmH and ptmQ double mutant | Disclosed |
| | | herein |
| S. pactum ΔptmB/TMW051 | ptmB mutant complementation with pTMW051 | Disclosed |
| | | herein |
| S. pactum ΔptmD/TMW052 | ptmD mutant complementation with pTMW052 | Disclosed |
| | | herein |
| S. pactum ΔptmH/TMW057 | ptmH mutant complementation with pTMW057 | Disclosed |
| | | herein |
| S. pactum ΔptmL/TMW053 | ptmL mutant complementation with pTMW053 | Disclosed |
| | | herein |
| S. pactum ΔptmM/TMW054 | ptmM mutant complementation with pTMW054 | Disclosed |
| | | herein |
| Staphylococcus aureus ATCC | Marker strain for antibacterial assay | ATCC |
| 12600 | | |
| Bacillus subtilis ATCC 6081 | Marker strain for antibacterial assay | ATCC |
| Pseudomonas aeruginosa ATCC | Marker strain for antibacterial assay | ATCC |
| 9721 | | |
| E. coli ATCC 11775 | Marker strain for antibacterial assay | ATCC |
| Plasmids | | |
| pBlueScript II SK(−) | ColE1-based phagemid vector with f1 (−) and pUC origins; | Stratagene |
| | T3, T7 and lac promoters; bla. | |
| pGEM-T | High copy number PCR cloning vector containing T7 and | Promega |
| | SP6 RNA polymerase promoters flanking a multiple | |
| | cloning region within the alpha-peptide coding region of the | |
| | enzyme beta-galactosidase; bla. | |
| pHZ1358 | tsr, bla, oriT, ori(pIJ101) | (2) |
| pOJ446 | E. coli-Streptomyces shuttle cosmid for conjugal transfer; | (3) |
| | aac(3)IV | |
| pJTU1278+ | pHZ1358 derivative containing lacZ and MCS | (4) |
| pTMN002 | pJTU1278+ derivative containing a 1 kb aac(3)IV | (5) |
| | apramycin resistance cassette from pOJ446 | |
| pTMW018 | pHZ1358 derivative containing oriT transfer element, the | (5) |
| | thiostrepton resistant gene thio^{R}, and the apramycin | |
| | resistance gene aac(3)IV | |
| pTMW025 | Two 1 kb PCR fragments upstream and downstream of the | Disclosed |
| | ptmB gene in pTMW018 | herein |
| pTMN003 | Two 1 kb PCR fragments upstream and downstream of the | Disclosed |
| | ptmD gene in pTMN002 | herein |
| pTMW026 | Two 1 kb PCR fragments upstream and downstream of the | Disclosed |
| | ptmH gene in pTMN002 | herein |
| pTMW027 | Two 1 kb PCR fragments upstream and downstream of the | Disclosed |
| | ptmL gene in pTMN002 | herein |
| pTMN004 | Two 1 kb PCR fragments upstream and downstream of the | Disclosed |
| | ptmM gene in pTMN002 | herein |
| pTMN005 | Two 1 kb PCR fragments upstream and downstream of the | Disclosed |
| | ptmY gene in pTMN002 | herein |
| pTMN101 | 1 kb aac(3)IV apramycin resistance cassette in ptmQ gene | (5) |
| pTMN001 | ptmQ-aac(3)IV fragment from pTMN101 in pHZ1358 | (5) |
| pSET152 | lacZα, ori(pUC18), oriT(RP4), int-attP(φC31), aac(3)IV | (3) |
| pWUX12a | pSET152 derivative containing ampicillin resistance gene | Disclosed |
| | bla | herein |
| pJTU695 | tsr, bla, oriT, ori, pI J101 derivative, PermE* | (6) |
| pTMW038 | pJTU695 derivative containing the apramycin resistance | Disclosed |
| | gene aac(3)IV | herein |
| pTMW051 | pWUX12a containing complete structural gene of ptmB | Disclosed |
| | | herein |
| pTMW052 | pWUX12a containing complete structural gene of ptmD | Disclosed |
| | | herein |
| pTMW057 | pWUX12a containing complete structural gene of ptmH | Disclosed |
| | | herein |
| pTMW053 | pWUX12a containing complete structural gene of ptmL | Disclosed |
| | | herein |
| pTMW054 | pWUX12a containing complete structural gene of ptmM | Disclosed |
| | | herein |
| TIP3 | Fosmid clone containing part of the ptm cluster | (5) |
| 5A7 | Fosmid clone containing part of the ptm cluster | (5) |

(1) Paget et al., J. Bacteriol, 1999, 181: 204-211
(2) Sun et al., Microbiology, 2002, 148: 361-371
(3) Kieser et al., The John Innes Foundation 2000, Norwich, England
(4) He et al., J. Microbiol. Biotechnol., 2010, 20: 678-682
(5) Ito et al., ChemBioChem 2009, 10, 2253-2265
(6) Bai et al., Chem. Biol. 2006, 13, 387

General DNA Manipulations:

Genomic DNA of S. pactum ATCC 27456 was prepared by standard protocol (Kieser et al., The John Innes Foundation 2000, Norwich, England) or using the DNeasy Tissue Kit (QIAGEN). DNA fragments were recovered from an agarose gel by using the QIAquick Gel Extraction Kit (QIAGEN). Restriction endonucleases were purchased from Invitrogen or Promega. Preparation of plasmid DNA was done by using a QIAprep Spin Miniprep Kit (QIAGEN). All other DNA manipulations were performed according to standard protocols (Kieser et al., The John Innes Foundation 2000, Norwich, England; Sambrook and Russell, Molecular Cloning: A Laboratory Manual, 2001, New York, Cold Spring Harbor Laboratory Press). PCR was performed in 30 cycles by using a Mastercycler gradient thermocycler (Eppendorf) and Platinum Taq DNA polymerase (Invitrogen, Carlsbad, Calif.) or Platinum Pfx DNA polymerase (Invitrogen). Oligodeoxyribonucleotides for PCR primers were synthesized by Sigma-Genosys, and are shown in Table 3. The nucleotide sequences of the gene fragments were determined at the Center for Genome Research and Biocomputing (CGRB) Core Laboratories, Oregon State University. ORFs were analyzed by FramePlot analysis (Ishikawa and Hotta, FEMS Microbiol. Lett., 1999, 174:251-253) and BLAST programs (Altschul et al., J. Mol. Biol., 1990, 215:403-410).

TABLE 3

| Primer | Sequence^{(a)} | SEQ ID NO: |
|---|---|---|
| ptmB-F1 | 5′-CCCAAGCTTCGTCGTAGCGTTCCAGCAGC-3′ | 1 |
| ptmB-R1 | 5′-CCGGAATTCGAGACCCAGAGCCAGCATACA-3′ | 2 |
| ptmB-F2 | 5′-CCGGAATTCTTCCTGGAGGACTTCCTGGTGG-3′ | 3 |
| ptmB-R2 | 5′-TGCTCTAGACGGTGAAGAAGGTGAGGTCGC-3′ | 4 |
| ptmB-P1 | 5′-ACATTTCGTAGTGCTGGTGCTTG-3′ | 5 |
| ptmB-P2 | 5′-GATGGTGTCCACGTCCTCGTC-3′ | 6 |
| ptmB-C1 | 5′-ATCATATGCTGGCTCTGGGTCTCGGCG-3′ | 7 |
| ptmB-C2 | 5′-CGGAATTCTCACTCGGTCTCCTCCGTC-3′ | 8 |
| ptmD-F1 | 5′-CCCAAGCTTGCTGAAGGGCTCCACCGA-3′ | 9 |
| ptmD-R1 | 5′-CCGGAATTCGGCCGAAATCCCGTCCAC-3′ | 10 |
| ptmD-F2 | 5′-CCGGAATTCGTCGGCCGGTTCAGCTGA-3′ | 11 |
| ptmD-R2 | 5′-TGCTCTAGACAGGAGCTGAAGGAGATC-3′ | 12 |
| ptmD-P1 | 5′-GCGACGGACAGAACCGCCTCAA-3′ | 13 |
| ptmD-P2 | 5′-ACGCCCTGCTGGTCTCCTTCCA-3′ | 14 |
| ptmD-C1 | 5′-CGCATATGATATCCGTGGACGGGATTTCGGC-3′ | 15 |
| ptmD-C2 | 5′-TAGAATTCTCAGCTGAACCGGCCGACG-3′ | 16 |
| ptmH-F1 | 5′-CCCAAGCTTGCTCGGCGTAGTAGGGGTCC-3′ | 17 |
| ptmH-R1 | 5′-CCGGAATTCTCCCCAGACGCCTTGCTGTA-3′ | 18 |
| ptmH-F2 | 5′-CCGGAATTCCCCAACACCGAGTACCACGA-3′ | 19 |
| ptmH-R2 | 5′-TGCTCTAGACGTCGCTTCCATCTTCGTCA-3′ | 20 |
| ptmH-P1 | 5′-TGAAGCCCAGCAGCTCCACC-3′ | 21 |
| ptmH-P2 | 5′-CGACCAGGATGACCTCGAACC-3′ | 22 |
| ptmH-C1 | 5′-ATCATATGGCCCCATTGCGTGGGAGAT-3′ | 23 |
| ptmH-C2 | 5′-ATGGATCCAATTGTCAAACGCGCGTGAACTGGTG-3′ | 24 |
| ptmL-F1 | 5′-CCCAAGCTTTCGGGGAGATCGCCCTGGAA-3′ | 25 |
| ptmL-R1 | 5′-CCGGAATTCCACCTTCTCGTAGAACAGGC-3′ | 26 |
| ptmL-F2 | 5′-CCGGAATTCGTCAGCGACCGCAAGGAGGA-3′ | 27 |
| ptmL-R2 | 5′-TGCTCTAGAGTGGATCAGGACGTCACGGG-3′ | 28 |
| ptmL-P1 | 5′-CCTTCTGGCGGAACCTGCTC-3′ | 29 |
| ptmL-P2 | 5′-GTGGATCAGGACGTCACGGG-3′ | 30 |
| ptmL-C1 | 5′-TACATATGGCTGACGCGGTGCTGCTGACGC-3′ | 31 |
| ptmL-C2 | 5′-ATGAATTCTCATCCGGCCAGGGCCCGTG-3′ | 32 |
| ptmM-F1 | 5′-CCCAAGCTTGCTACAACCACGCCGTGG-3′ | 33 |
| ptmM-R1 | 5′-CCGGAATTCGGGTGTCAGCAGGACCAC-3′ | 34 |
| ptmM-F2 | 5′-CCGGAATTCCTGATCAACCGGGCCATC-3′ | 35 |
| ptmM-R2 | 5′-TGCTCTAGAGCGGAACGGCATGTTCAC-3′ | 36 |
| ptmM-P1 | 5′-CTTCTGCTACGTGCCGCGGGCCC-3′ | 37 |
| ptmM-P2 | 5′-GCGGTCAGCGAGTGGCAGCGGAACG-3′ | 38 |
| ptmM-C1 | 5′-TTCATATGTCGGACGTGGTCCTGCTGACAC-3′ | 39 |
| ptmM-C2 | 5′-ATGAATTCTCAGCCCCTCACCTGTGCCA-3′ | 40 |
| ptmY-F1 | 5′-CCCAAGCTTGCGCACACCATGTACCAG-3′ | 41 |
| ptmY-R1 | 5′-CCGGAATTCGAGGGTGGGGGTGTGCAT-3′ | 42 |
| ptmY-F2 | 5′-CCGGAATTCCTGATGCGCGGCCTGCAC-3′ | 43 |
| ptmY-R2 | 5′-TGCTCTAGAGCTCGCCTACTACGACAG-3′ | 44 |
| ptmY-P1 | 5′-CGGCGGCGACCTCACCTTCTTC-3′ | 45 |
| ptmY-P2 | 5′-CCCCGGAACAGCCGCAGGACGG-3′ | 46 |

^{(a)}nucleotides in bold refer to restriction sites

Analysis of Metabolites from the ptmB, ptmD, ptmH, ptmL, ptmM, PtmY, and ptmH/ptmQ Mutants:

The mutant strains of S. pactum were grown on BTT agar [glucose (1%), yeast extract (0.1%), beef extract (0.1%), casein hydrolysate (0.2%), agar (1.5%), pH 7.4] at 30° C. for 3 days. Single colonies were used to inoculate the BTT seed cultures and incubated at 30° C. for 3 days. Production cultures were prepared in modified Bennet medium (Sakuda et al., J. Org. Chem. 2001, 66, 3356) (50 mL) and inoculated with seed cultures [10% (V/V)]. The production cultures were incubated at 30° C. for five days under vigorous shaking (200 rpm). The mycelia were centrifuged and the supernatants were extracted twice with equal volumes of ethyl acetate. The organic solvent was evaporated in vacuo and the residues were dissolved in MeOH and analyzed by reverse-phase HPLC or LC-MS. HPLC analyses were carried out using a C-18 silica gel column (Phenomenex Gemini 5μ, 4.6×150 mm) and 5 mM NH₄OAc solution (solvent A, 40%) and MeOH/MeCN (4:1) (solvent B, 60%) as the mobile phase at a flow rate of 0.2 mL/min and UV detection at 240 nm. The molecular weight of each compound was determined by electrospray mass spectrometry.

Complementation of the ptmB, ptmD, ptmH, ptmL, and ptmM Mutants:

The ptmB, ptmD, ptmH, ptmL, and ptmM genes were individually cloned into pTMW038, which was derived from pJTU695 (Bai et al., Chem. Biol., 2006, 13:387-397) by inserting the apramycin resistance gene aac(3)IV, and the products were transferred into the corresponding mutant strains by conjugation. Analysis of the culture broths of the conjugant strains, however, revealed that the expression system did not work properly in S. pactum. No recovery of pactamycin production was observed in all tested conjugants. Therefore, the genes together with the ermE* promoter were retrieved from the plasmids and introduced into the EcoRV/EcoRI site of pWUX12a, which was derived from the integrative vector pSET152 containing the ampicillin resistance gene (bla). The new plasmids, pTMW051, pTMW052, pTMW057, pTMW053, and pTMW054, were then introduced into the ΔptmB, ΔptmD, ΔptmH, ΔptmL, and ΔptmM strains, respectively, and pactamycin production was examined by mass spectrometry (MS).

Construction of ptmB, ptmD, ptmH, ptmL, ptmM, ptmY, and ptmH/ptmQ Knockout Mutants:

Two ˜1 kb PCR fragments upstream and downstream of the genes were fused and cloned into the HindIII/XbaI site of pBluescript II SK(−) vector. For ptmB, the products were subsequently excised with HindIII and XbaI, treated with Klenow enzyme, and ligated into pTMW018 (predigested with BamHI and treated with Klenow enzyme) to create pTMW025 (Table 1). For ptmD, ptmH, ptmL, ptmM, and ptmY, the products were excised and cloned into the HindIII/XbaI site of pTMN002 to create pTMN003, pTMW026, pTMW027, pTMN004, and pTMN005, respectively. All plasmids were then individually introduced into S. pactum ATCC 27456 by conjugation using the E. coli donor strain ET12567/pUZ8002. For ptmH/ptmQ mutant, the plasmid pTMN001 (Ito et al., Chembiochem., 2009, 10:2253-2265) was transferred into E. coli ET12567/pUZ8002, and subsequently introduced into the ΔptmH strain by conjugation. Screening and analysis of the mutants were performed as described in Example 2.

Production and Purification of de-6MSA-7-demethyl-7-deoxypactamycin (TM-025) and 7-demethyl-7-deoxypactamycin (TM-026):

The mutant strains of S. pactum were cultivated in the production medium (modified Bennet medium) (Ito et al., 2009). The supernatant from 10 L of culture was extracted three times with equal volumes of ethyl acetate. The organic solvent was evaporated in vacuo to give a yellow solid, which was subsequently dissolved in a small amount of ethyl acetate-toluene (2:1). The sample was loaded onto a silica gel column (1.4 cm×20 cm), which was eluted with ethyl acetate-toluene (2:1) (250 mL) and then with methanol (600 mL) to give fractions that contain TM-025 or TM-026. For TM-025, fractions containing the compound were pooled and evaporated to dryness, and the product was further purified by HPLC using a C-18 silica gel column (YMC-pack ODS-A, 4.6×250 mm, Kyoto, Japan) and 0.015 M NH₄OAc buffer (pH 5.5)-MeOH (65:35) as the mobile phase at a flow rate of 2.2 mL/min and UV detection at 240 nm to give pure TM-025 17 mg. Similar HPLC conditions were used for the purification of TM-026, except that the mobile phase was 0.015 M NH₄OAc buffer (pH 5.5)-MeOH-MeCN (40:48:12) and the flow rate was 0.9 mL/min to yield 150 mg of TM-026.

Structure Characterization of TM-025, TM-026:

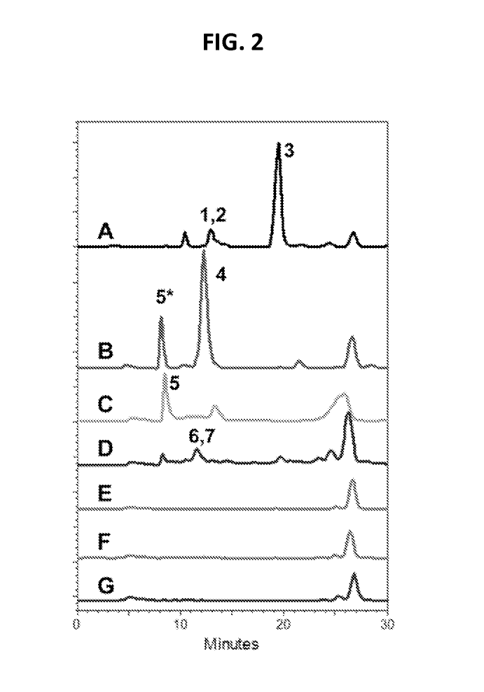

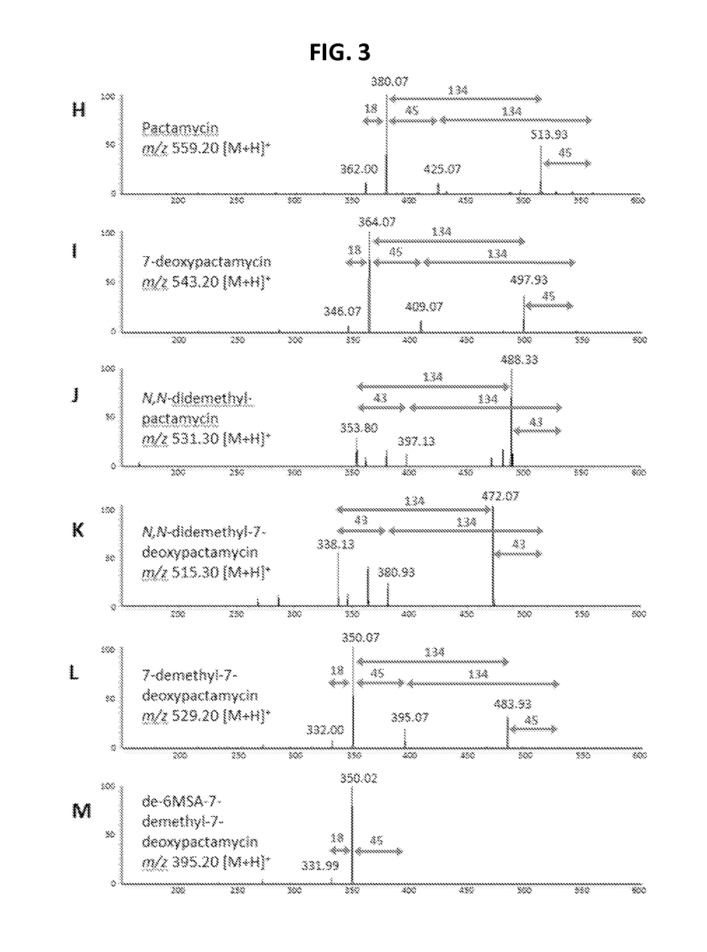

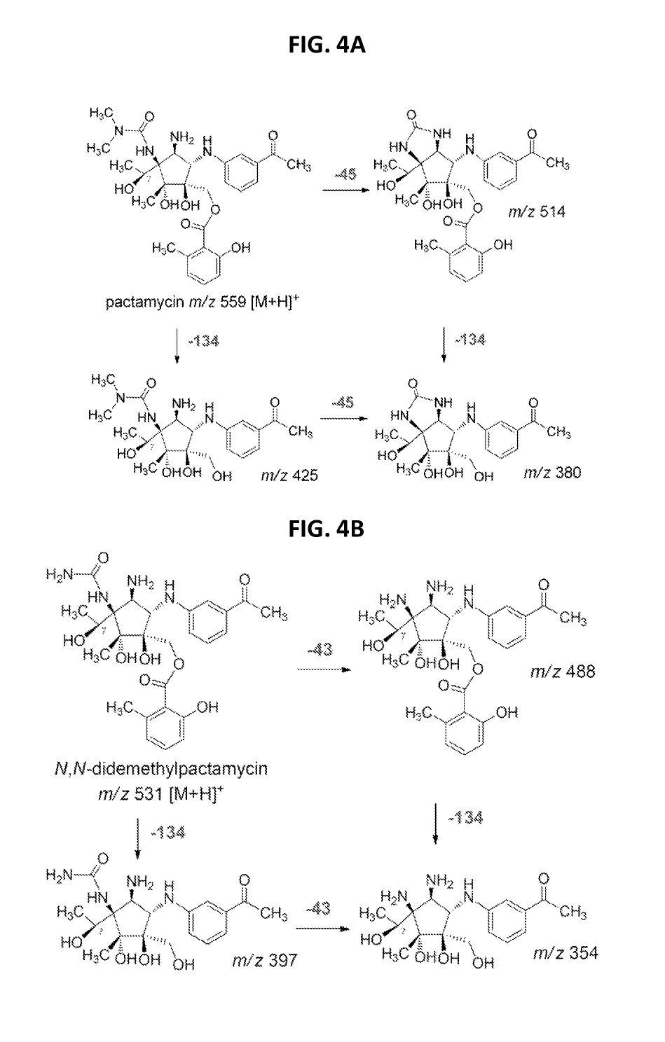

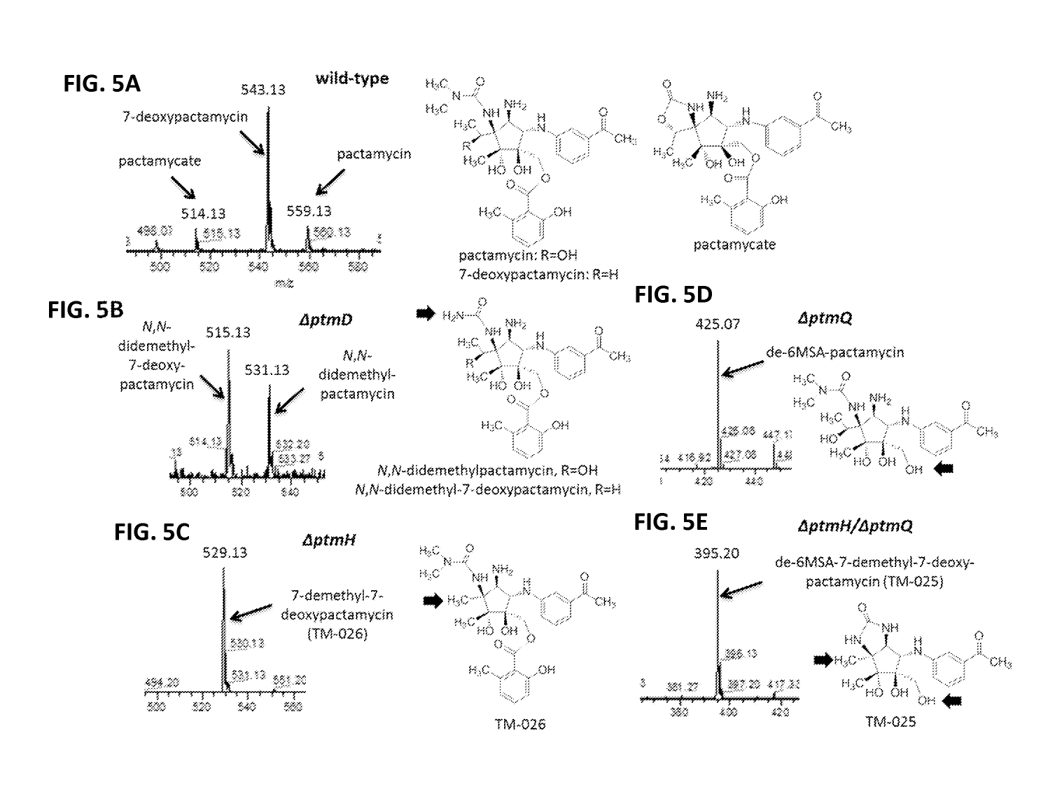

To fully elucidate the chemical structures of TM-025 and TM-026, the compounds were subjected to HPLC, tandem mass spectrometric and NMR spectroscopic analyses (FIGS. 2, 3. 4A-4B, 5A-5E, 6A-6D, 7A-7D). ESI MS/MS analysis of TM-026 gave fragment ions m/z 483.93, 395.07, 350.07, and 332.00, which are consistent with the cleavage of the dimethylamino (45 amu) and the 6-methylsalicylic acid (134 amu) moieties, as well as the loss of water (18 amu) (FIG. 3, spectrum L). On the other hand, the fragment ions of TM-025 only showed the cleavage of the dimethylamino group and the loss of water (FIG. 3, spectrum M), indicating that there is no 6-methylsalicylic acid moiety in TM-025.

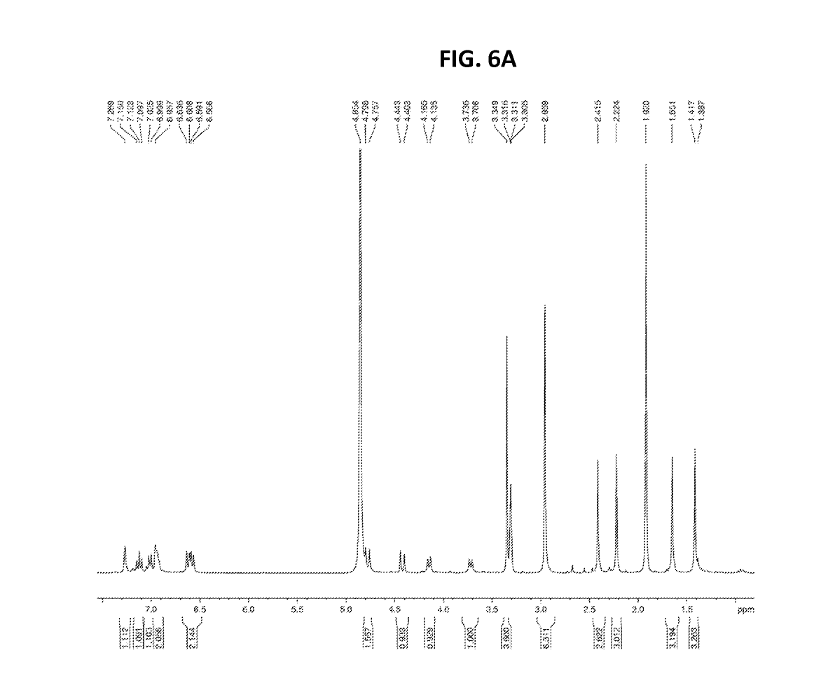

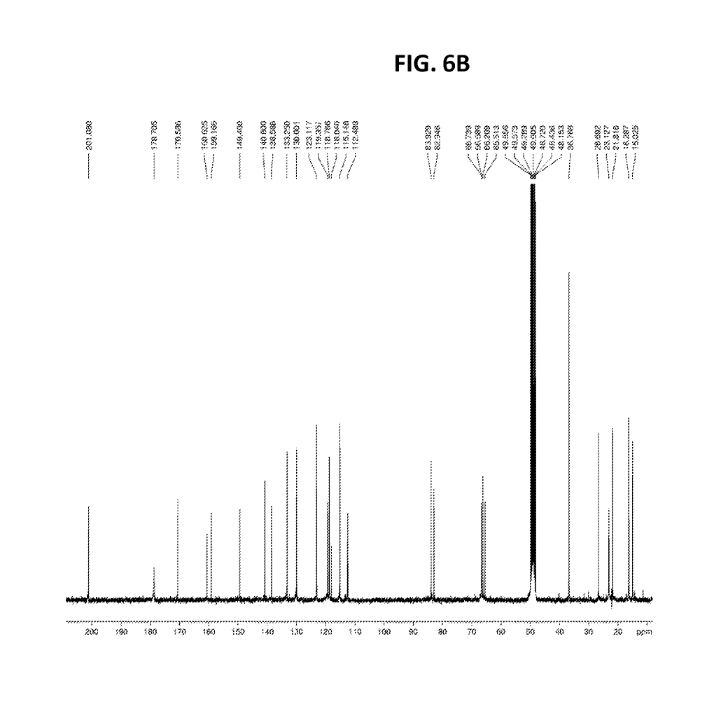

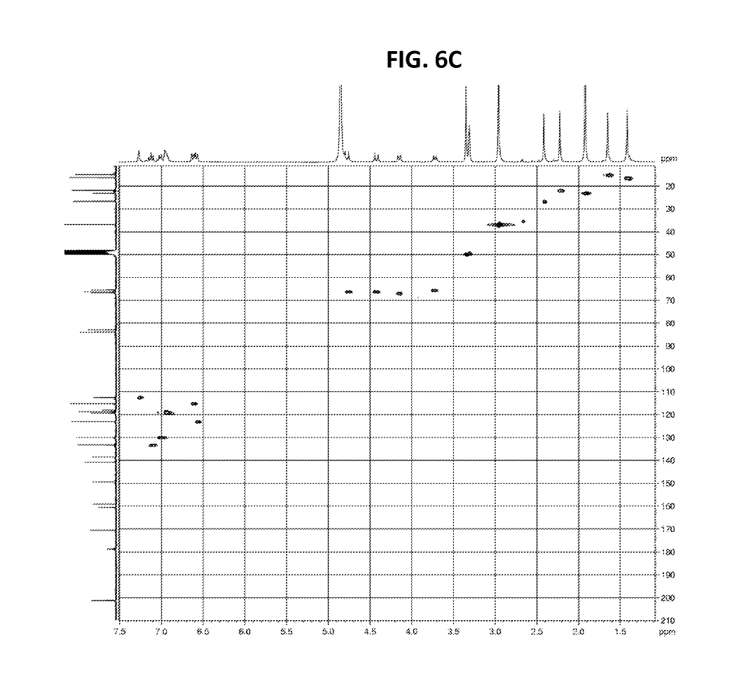

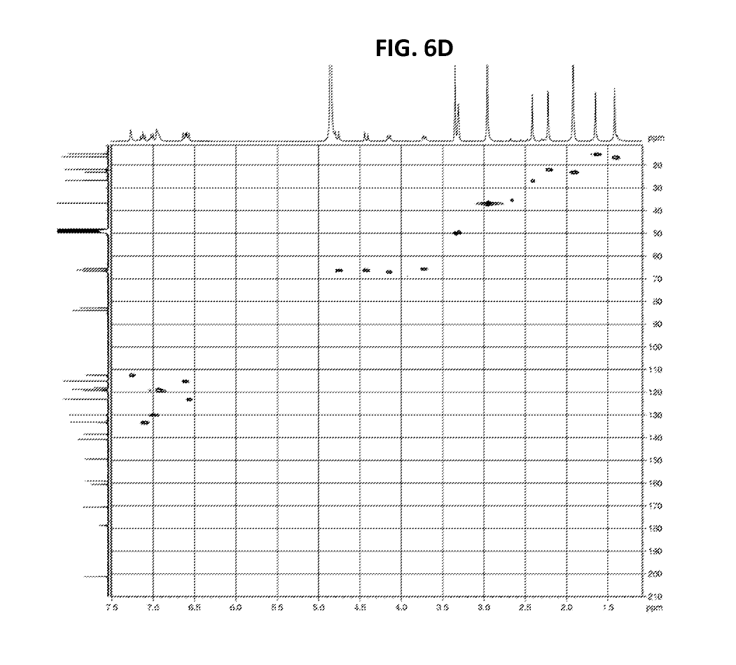

The ¹H NMR spectrum of TM-026 (FIG. 6A) showed signals similar to those of pactamycin, except that signals corresponding to the C-7 and C-8 hydroxyethyl unit in pactamycin are missing. In addition, a new methyl signal (δ_{H} 1.65 ppm) is observed in the ¹H NMR spectrum of TM-026, suggesting that the hydroxyethyl unit in pactamycin has been replaced by a methyl group in TM-026.

The ¹³C NMR spectrum of TM-026 (FIG. 6B) showed 27 carbon signals. Based on the DEPT135 spectrum and confirmed by the HMBC and HSQC spectra (FIGS. 6C and 6D, respectively), there are fifteen methyl and methine, one methylene, and eleven quaternary carbons, which are consistent with those expected for TM-026.

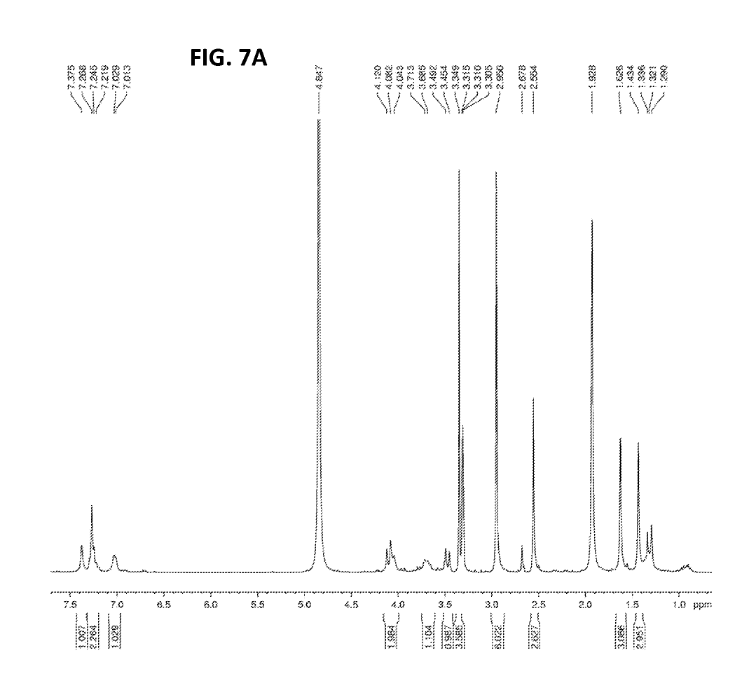

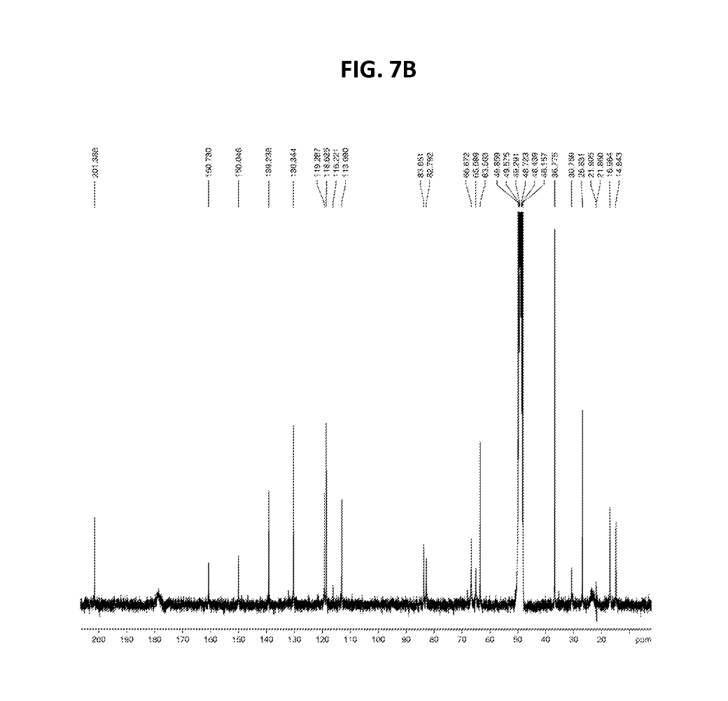

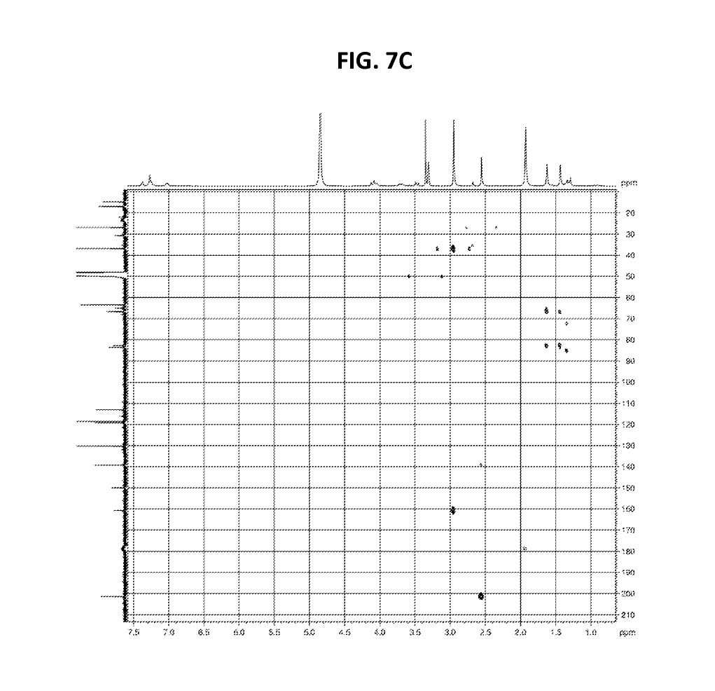

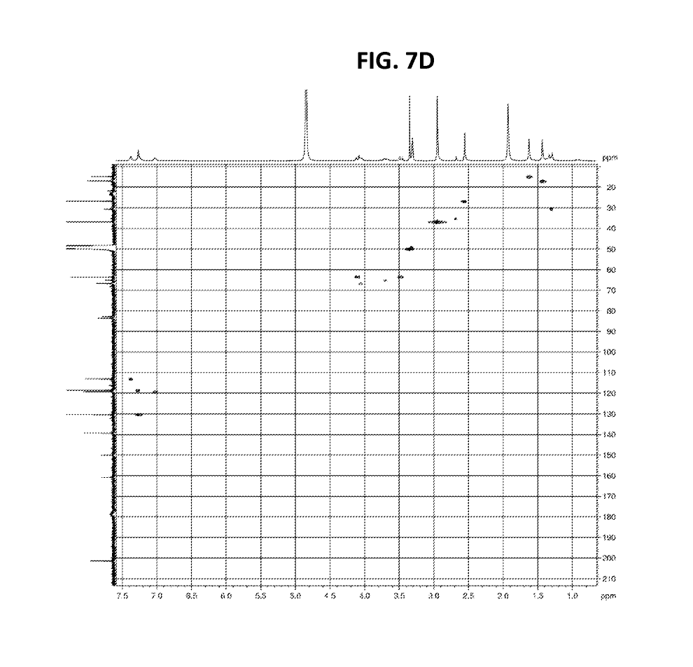

The ¹H and ¹³C NMR spectra of TM-025 (FIGS. 7A and 7B, respectively) showed signals similar to those of de-6MSA-pactamycin, with the exception that no signals related to the hydroxyethyl functionality were observed. As in TM-026, the missing hydroxyethyl moiety is replaced by a methyl group (δ_{H} 1.63 ppm, δ_{C} 14.8 ppm). In addition, the C-7′ methyl signal and some signals in the aromatic region are missing, indicating the absence of 6MSA in TM-025. This is supported by the upfield shifts of the C-8 methylene proton signals from 4.42 (d, J=12 Hz) and 4.78 (d, J=12 Hz) in TM-026 to 3.47 (d, J=12 Hz) and 4.10 (d, J=12 Hz) in TM-025, and of the C-8 methylene carbon from 66.2 ppm in TM-026 to 63.5 ppm in TM-025. The ¹³C NMR spectrum of TM-025 (FIG. 7B) showed 18 carbons, one signal less than expected for the 19 carbons of TM-025. However, based on HMBC and HSQC studies (FIGS. 7C and 7D, respectively), the missing signal was identified to be that of C-1 at δ_{C} 66.7 ppm, overlaps with the C-3 signal. Altogether the data provided convincing evidence that TM-026 is 7-demethyl-7-deoxypactamycin and TM-025 is de-6MSA-7-demethyl-7-deoxypactamycin.

TM-025:

Yellowish powder, ¹H NMR (300 MHz, CD₃OD): δ7.40 (1H, br s, H-2″), 7.25 (2H, m, H-4″, H-5″), 7.01 (1H, m, H-6″), 4.10 (1H, d, J=12 Hz, H-8a), 4.06 (1H, br d, J=9 Hz, H-3), 3.70 (1H, br d, J=9 Hz, H-2), 3.47 (1H, d, J=12 Hz, H-8b), 2.95 (6H, s, N(CH₃)₂), 2.55 (3H, s, —COCHH₃), 1.63 (3H, s, 7-CH₃), 1.43 (3H, s, 6-CH₃). ¹³C NMR (75.5 MHz, CD₃OD): δ201.4 (s, C-7″), 160.7 (s, C-9), 150.0 (s, C-3″), 139.2 (s, C-1″), 130.3 (d, C-5″), 119.3 (d, C-6″), 118.6 (d, C-4″), 113.1 (d, C-2″), 83.7 (s, C-4), 82.8 (s, C-5), 66.7 (s, C-1), 66.7 (d, C-3), 65.1 (d, C-2), 63.5 (t, C-8), 36.8 (q, C-10 and C-11), 26.8 (q, C-8″), 17.0 (q, C-6), 14.8 (q, C-7). (+)-ESIMS: m/z=395 [M+H]⁺. HR-ESIMS m/z 395.2314 [M+H]⁺, calculated for C₁₉H₃₁N₄O₅ m/z 395.2294.

TM-026:

Yellowish powder, ¹H NMR (300 MHz, CD₃OD): δ7.27 (1H, br s, H-2″), 7.12 (1H, t, J=8 Hz, H-4′), 7.01 (1H, t, J=8 Hz, H-5″), 6.96 (1H, m, H-4″, H-6″), 6.62 (1H, d, J=8 Hz, H-3′), 6.58 (1H, d, J=8 Hz, H-5′), 4.78 (1H, d, J=12 Hz, H-8a), 4.42 (1H, d, J=12 Hz, H-8b), 4.15 (1H, d, J=9 Hz, H-3), 3.72 (1H, d, J=9 Hz, H-2), 2.96 (6H, s, N(CH₃)₂), 2.42 (3H, s, —COCH₃), 2.22 (3H, s, 7′-CH₃), 1.65 (3H, s, 7-CH₃), 1.42 (3H, s, 6-CH₃). ¹³C NMR (75.5 MHz, CD₃OD): δ 201.1 (s, C-7″), 170.6 (s, C-8′), 160.6 (s, C-9), 159.2 (s, C-2′), 149.4 (s, C-3″), 140.8 (s, C-6′), 138.6 (s, C-1″), 133.3 (d, C-4′), 130.0 (d, C-5″), 123.1 (d, C-5′), 119.4 (d, C-6″), 118.8 (d, C-4″), 118.0 (s, C-1′), 115.1 (d, C-3′), 112.5 (d, C-2″), 83.9 (s, C-4), 82.9 (s, C-5), 66.7 (s, C-1), 66.6 (d, C-3), 66.2 (t, C-8), 65.5 (d, C-2), 36.8 (q, C-10, C-11), 26.7 (q, C-8″), 21.8 (q, C-7′), 16.3 (q, C-6), 15.0 (q C-7). (+)-ESIMS: m/z=529 [M+H]⁺. HR-ESIMS m/z 529.2670 [M+H]⁺, calculated for C₂₇H₃₇N₄O₇ m/z 529.2662.

Antimalarial Activity Assay:

An antimalarial activity assay was carried out at the Oregon Translational Research and Drug Development Institute (OTRADI). Two strains of P. falciparum were used. The chloroquine-sensitive clone D6, and chloroquine-resistant and MDR clone Dd2 was obtained from the Malaria Research and Reference Reagent Resource Center (MR4) (Manassas, Va.). The parasites were cultured according to the method of Trager and Jensen (Trager and Jensen, Science, 1976, 193:673-675), with minor modifications. The cultures were maintained in human erythrocytes (Lampire Biological Laboratories, Pipersville, Pa.), suspended at 2% hematocrit in RPMI 1640 (Sigma) containing 0.5% Albumax (Invitrogen), 45 μg/L hypoxanthine (Lancaster), and 50 μg/L gentamicin (Invitrogen), and incubated at 37° C. under a gas mixture of 5% O₂, 5% CO₂, and 90% N₂. In vitro antimalarial activity was determined by a SYBR Green I fluorescence-based method described previously by Smilkstein et al. (Smilkstein et al., Antimicrob. Agents Chemother., 2004, 48:1803-1806). Frozen stock solutions of each test drug (1 mM in DMSO) were thawed, warmed to 37° C., and mixed thoroughly prior to use. Drug solutions were serially diluted with culture medium, and distributed to asynchronous parasite cultures on 96-well plates in quadruplicate in a total volume of 100 μL to achieve 0.2% parasitemia with a 2% hematocrit in a total volume of 100 μL. Automated pipeting and dilution was carried using programmable Precision XS (Bio-Tek, Winooski, Vt.) and Sciclone ALH3000 (Caliper, Hopkinton, Mass.) robotic stations. The plates then were incubated for 72 hours at temperature and gas conditions described above. After incubation, 100 μL of lysis buffer with SYBR Green I was added to each well. The plates were incubated at room temperature for 1 h and then placed in a 96-well fluorescence plate reader (Synergy4, BioTek, Winooski, Vt.) with excitation and emission wavelength at 485 nm and 528 nm, respectively, for measurement of fluorescence. The 50% inhibitory concentration (IC₅₀) was determined by non-linear regression analysis of logistic dose-response curves (GraphPad Prism software).

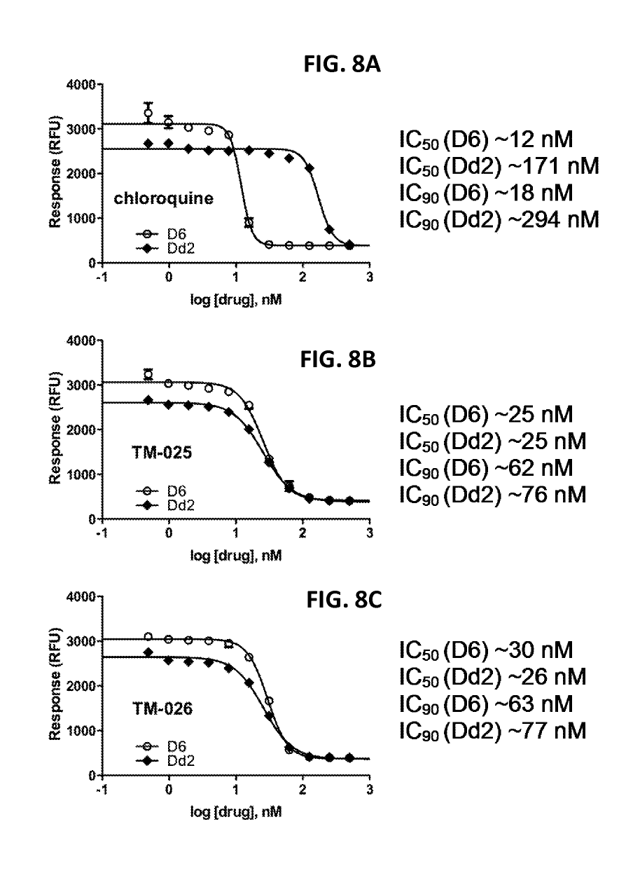

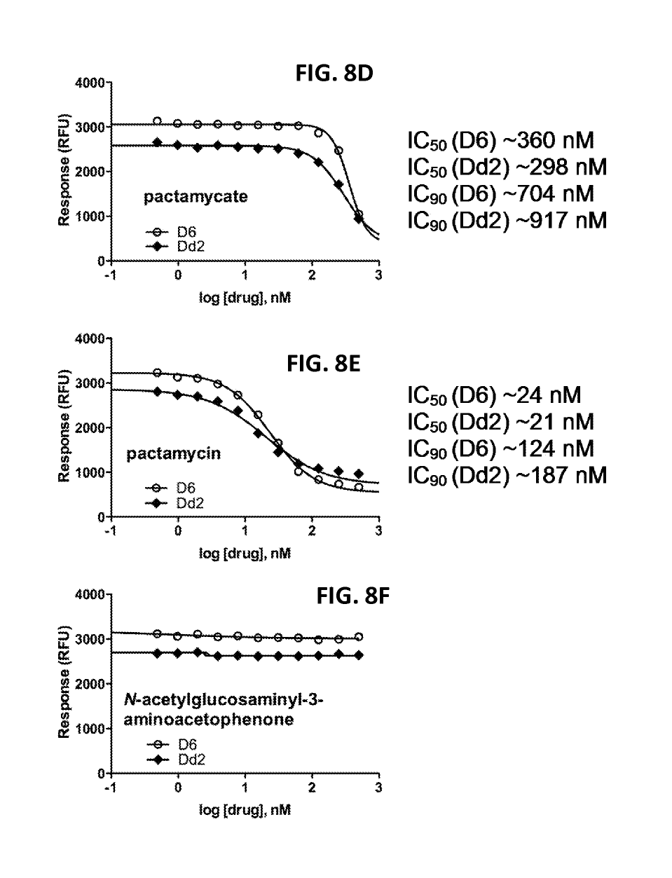

The new compounds TM-025 and TM-026 showed pronounced antimalarial activity with IC₅₀ between 25-30 nM against both strains, on a par with pactamycin (FIGS. 8A-8F). However, the IC₉₀ values for TM-025 and TM-026 (62-77 nM) (FIGS. 8B and 8C, respectively) were significantly lower than for pactamycin (124 nM and 187 nM) (FIG. 8E), particularly against the Dd2 strain, indicating superiority of the new analogs in their antimalarial activity over the parent compound. The results also indicate a possible acquired resistance within the parasites against pactamycin (FIG. 8E). As a comparison, the less active analog pactamycate showed IC₅₀ and IC₉₀ at around 300 nM and 800 nM, respectively, against both strains (FIG. 8D). Moreover, the inhibitory patterns of TM-025 and TM-026 appeared to be somewhat different from that of pactamycin, which indicates possibly that these analogs inhibit plasmodial growth via a distinct mechanism of action. The putative pactamycin biosynthetic precursor N-acetylglucosaminyl-3-aminoacetophenone was also evaluated (FIG. 8F), and its lack of activity suggests that the antimalarial activity is not due to the aminoacetophenone moiety.

Antibacterial Activity Assay:

The antibacterial activity of pactamycin and its analogs was determined by agar diffusion and micro-dilution assays. E. coli, P. aeruginosa, B. subtilis or M. smegmatis, and S. aureus were streaked on nutrient agar (Difco or Beckton-Dickinson) and grown overnight at 37° C. Colonies were transferred to nutrient broth and incubated at 37° C. for 24 hours. Turbidity of the inoculum was measured to a proper density at 600 nm (BioRad, SmartSpec 3000). For plate preparation, inoculum (500 μL) was mixed thoroughly with warm nutrient agar (50 mL) and poured to 25 mL square plates. The agar plates were allowed to solidify and dry for 30 min before assay. Sterile blank paper disks (Becton-Dickinson) were impregnated with pactamycin and its analogs (20 μL) at various concentrations and dried at room temperature. The disks were placed onto inoculated agar plates and incubated at 37° C. for 24 hours. In order to produce a contrast background of the inhibition zone, 0.25% MTT developing dye (1 mL) was added over the plates.

Micro-dilution assays were performed in 96-well plates. The compounds were serially diluted and added to the bacterial suspension at final concentrations of 1 mM-10 nM, or 0.5 mM-10 nM. After incubation at 37° C. for 24 hours, 0.25% MTT developing dye (50 μL) was added. Each assay was done in triplicate.

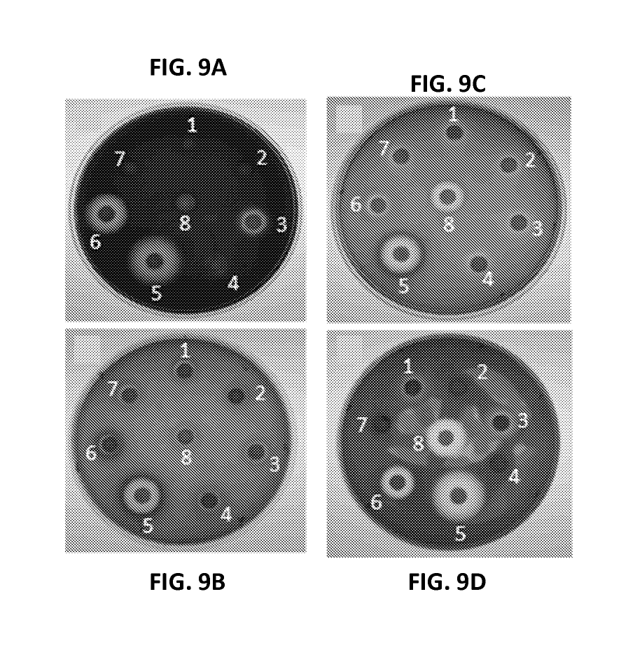

Surprising results emerged from the antibacterial assays. While pactamycin demonstrates a strong antibacterial activity against both Gram-positive and Gram-negative bacteria, neither TM-025 nor TM-026 showed any significant activity in the agar diffusion (FIGS. 9A-9D) and micro-dilution assays at the concentrations used. The IC₅₀ of pactamycin was about 10 μM against Staphylococcus aureus, 500 μM against Pseudomonas aeruginosa and Bacillus subtilis, and 100 μM against Escherichia coli, whereas the IC₅₀s of TM-025 and TM-026 were consistently higher (mostly >1 mM) against all tested bacterial strains. The results indicate that these analogs have less affinity to the bacterial ribosome or interact with it in a less damaging fashion than pactamycin.

Cytotoxicity:

HCT116 (colorectal carcinoma) cells were seeded onto a 96-well plate at 10,000 cells per well. Cells were treated with either 5, 50, 500 or 5000 nM TM-025, TM-026, N-acetyl-glucosaminyl-3-aminoacetophenone or 5, 25, 50, 100 nM pactamycin in triplicate for 48 hours. For the narrow range concentration assays, cells were treated with either 0.5, 1, 2 or 4 μM TM-025; 1, 2, 3 or 5 μM TM-026; 0.5, 2, 5 or 7 μM N-acetylglucosaminyl-3-aminoacetophenone or 12.5, 25, 50, 100 nM pactamycin in triplicate for 24 or 48 hours. WST-8 [2-(2-methoxy-4-nitrophenyl)-3-(4-nitrophenyl)-5-(2,4-disulfophenyl)-2H-tetrazolium, monosodium salt] (Dojindo, Rockville, Md.) was added to each well to give an orange-colored formazan product, which is soluble in tissue culture medium. Absorbance was measured at 450 nm. Percent of viable cells was calculated relative to the “No Treatment” and “Solvent Only” wells.

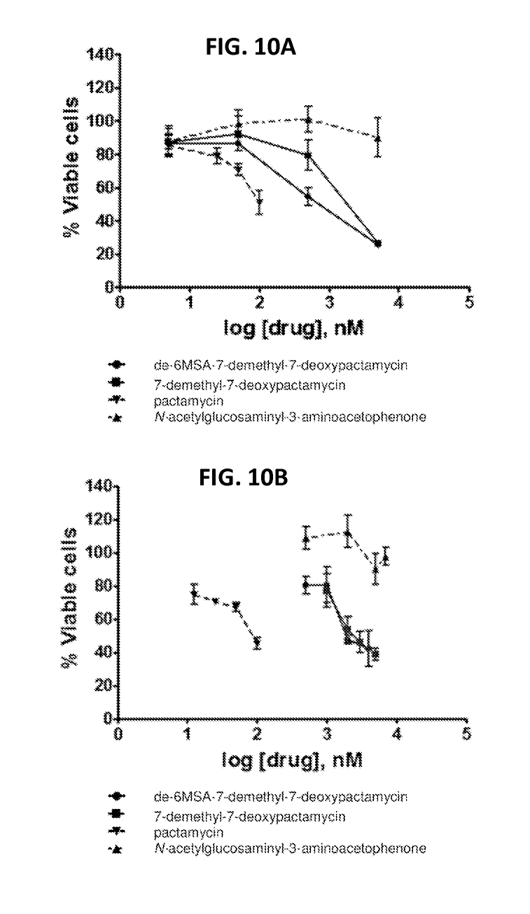

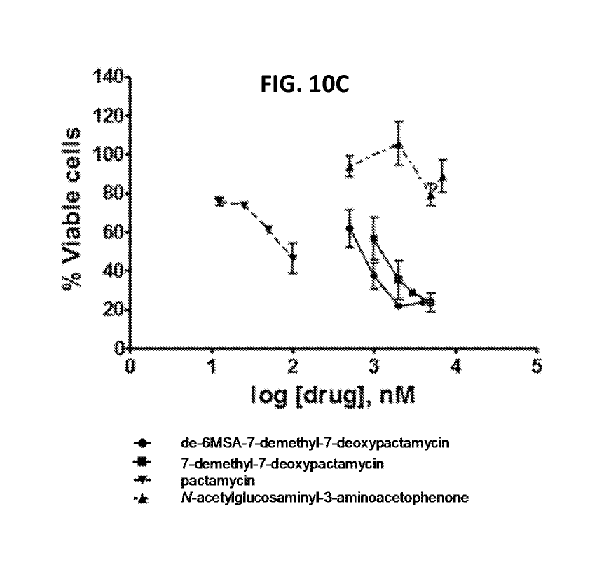

The results revealed that the evaluated analogs are significantly less toxic than pactamycin with estimated IC₅₀ values between 1,000 and 3,000 nM, or about 10-30 times higher than that of pactamycin (IC₅₀˜100 nM) (FIGS. 10A-10C). The results indicate that the analogs have less affinity towards the ribosome of mammalian cells, or that their antimalarial activity is due to a different mechanism of action. Table 4 summarizes the bioactivity comparisons between pactamycin, 7-demethyl-7-deoxypactamycin, and de-6MSA-7-demethyl-7-deoxypactamycin.

TABLE 4

| Compound | Antibacterial | Antimalarial | Cytotoxicity |
|---|---|---|---|
| Pactamycin | +++ | IC₅₀ (D6) ~24 nM | IC₅₀ ~100 nM |
| | | IC₅₀ (Dd2) ~21 nM | |
| | | IC₉₀ (D6) ~124 nM | |
| | | IC₉₀ (Dd2) ~187 nM | |
| 7-demethyl-7- | — | IC₅₀ (D6) ~30 nM | IC₅₀ ~3,000 nM |
| deoxy- | | IC₅₀ (Dd2) ~26 nM | |
| pactamycin | | IC₉₀ (D6) ~63 nM | |
| | | IC₉₀ (Dd2) ~77 nM | |
| de-6MSA-7- | — | IC₅₀ (D6) ~25 nM | IC₅₀ ~1,300 nM |
| demethyl-7- | | IC₅₀ (Dd2) ~25 nM | |
| deoxy- | | IC₉₀ (D6) ~62 nM | |
| pactamycin | | IC₉₀ (Dd2) ~76 nM | |

### Example 2

### Production and Analysis of de-6MSA-pactamycin and de-6MSA-pactamycate

This example describes the production and analysis of de-6MSA-pactamycin and de-6MSA-pactamycate.

Bacterial Strains and Plasmids:

Pactamycin producing S. pactum ATCC 27456 was purchased from American Type Culture Collection (ATCC). S. lividans T7 was used for heterologous expression of the 6MSA synthase gene. Escherichia coli DH10B was used as a host strain for the construction of recombinant plasmids. E. coli ET12567 was used as donor strain in conjugation studies. pBlueScript II (SK−) (Stratagene) and pGEM-T Easy (Promega) were used as cloning vectors. Construction of a genomic library of S. pactum was performed using pCC1FOS™ vector (EPICENTRE). pJTU780, a pRSET B derivative containing a MfeI site upstream of the T7 promoter region and pGM9 (Wohlleben et al., Acta Microbiol. Immunol. Hung. 1994, 41, 381) were used for construction of a shuttle plasmid for heterologous expression. pOJ446 was used for amplification of Apr^{R} gene (Kieser et al., The John Innes Foundation 2000, Norwich, England). pHZ1358 is a pIJ101 derivative containing an OriT transfer element required for conjugation (Sun et al., Microbiology 2002, 148, 361). Other bacterial strains and plasmids used in this study are listed in Table 5.

TABLE 5

| | Relevant genotype/comments | Source/Ref |
|---|---|---|
| Strains | | |
| Escherichia coli DH10B | F⁻ mcrA Δ(mrr-hsdRMS-mcrBC)φ80lacZΔM15 | GibcoBRL |
| | ΔlacX74 recA1 endA1 araD139 Δ(ara, leu)7697 galU | |
| | galK λ⁻ rspL nupG | |
| E. coli ET12567(pUZ8002) | dam dcm hsdS, pUZ8002 | [1] |
| E. coli ATCC 11775 | Marker strain for antibacterial assay | ATCC |
| Streptomyces pactum | Wild-type pactamycin producing strain | ATCC |
| ATCC 27456 | | |
| S. pactum MT-ptmC | ptmC knock-out mutant | Disclosed |
| | | herein |
| S. pactum MT-ptmJ | ptmJ knock-out mutant | Disclosed |
| | | herein |
| S. pactum PKSM1/3 | ptmQ knock-out mutant | Disclosed |
| | | herein |
| S. pactum PKSM1/8 | ptmQ knock-out mutant | Disclosed |
| | | herein |
| S. pactum PKSM1/19 | ptmQ knock-out mutant | Disclosed |
| | | herein |
| S. lividans T7 | Heterologous expression host | [2] |
| Mycobacterium smegmatis | Marker strain for antibacterial assay | ATCC |
| ATCC 14468 | | |
| Staphylococcus aureus | Marker strain for antibacterial assay | ATCC |
| ATCC 12600 | | |
| Pseudomonas aeruginosa | Marker strain for antibacterial assay | ATCC |
| ATCC 9721 | | |
| Amycolatopsis | Wild-type rifamycin producing strain | [3] |
| mediterranei S699 | | |
| Plasmids | | |
| pBlueScript II SK(−) | ColE1-based phagemid vector with f1 (−) and pUC | Stratagene |
| | origins; T3, T7 and lac promoters; bla. | |
| pGEM-T Easy | High copy number PCR cloning vector containing T7 | Promega |
| | and SP6 RNA polymerase promoters flanking a | |
| | multiple cloning region within the alpha-peptide | |
| | coding region of the enzyme beta-galactosidase; bla. | |
| pCC1FOS | contains both the E. coli F-factor single-copy origin | Epicenter |
| | of replication and the inducible high-copy oriV. | |
| pHZ1358 | tsr, bla, oriT, ori(pIJ101) | [4] |
| pJTU1278+ | pHZ1358 derivative containing lacZ and MCS | [5] |
| pTMN002 | pJTU1278+ derivative containing a 1 kb aac(3)IV | Disclosed |
| | apramycin resistance cassette from pOJ446 | herein |
| pTMW018 | pHZ1358 derivative containing the aac(3)IV | Disclosed |
| | apramycin resistance cassette and MCS from | herein |
| | pTMN002 | |
| pTMW028 | Two 1 kb PCR fragments upstream and downstream | Disclosed |
| | of the functional domain of ptmC in pTMW018 | herein |
| pTMW029 | Two 1 kb PCR fragments upstream and downstream | Disclosed |
| | of the functional domain of ptmJ in pTMW018 | herein |
| pOJ446 | E. coli-Streptomyces shuttle cosmid for conjugal | [6] |
| | transfer; Apr^{R} | |
| TIP3 | Fosmid clone containing part of the ptm cluster | Disclosed |
| | | herein |
| 5A7 | Fosmid clone containing part of the ptm cluster | Disclosed |
| | | herein |
| 28F2 | Fosmid clone containing part of the ptm cluster | Disclosed |
| | | herein |
| 24H11 | Fosmid clone containing part of the ptm cluster | Disclosed |
| | | herein |
| pJTU780 | pRSET B derivative containing MfeI site upstream of | Gift from |
| | the T7 promoter | Drs. |
| | | Deng/Bai |
| pTMT001 | 2.1 kb DNA fragment containing the ptmQ upstream | Disclosed |
| | sequence (ptmQf1) in pJTU780 | herein |
| pTMT002 | 1.3 kb DNA fragment containing the ptmQ center | Disclosed |
| | sequence (ptmQf2) in pJTU780 | herein |
| pTMT003 | 2.1 kb DNA fragment containing the ptmQ | Disclosed |
| | downstream sequence (ptmQf3) in pJTU780 | herein |
| pTMT004 | 3.4 kb DNA fragment containing the ptmQ upstream | Disclosed |
| | and center sequences (ptmQf1 + 2) in pJTU780 | herein |
| pTMT005 | 5.5 kb DNA fragment containing the whole ptmQ | Disclosed |
| | gene in pJTU780 | herein |
| pTMN101 | 1 kb DNA fragment containing the Apr^{R} gene in | Disclosed |
| | pTMT005 | herein |
| pTMN001 | 3.4 kb SalI/NcoI digested ptmQ-Apr^{R} fragment in | Disclosed |
| | pHZ1358 | herein |
| pGM9 | pSG5-derived temperature-sensitive replication | [7] |
| | vector, tsr aphII | |
| pTMN102 | fusion of the entire pTMT005 plasmid into pGM9 | Disclosed |
| | | herein |

[1] Paget et al. J. Bacterial. 1999, 181, 204.
[2] Heinzelmann et al., Microbiology 2005, 151, 1963.
[3] Xu et al., Microbiology 2005, 151, 2515.
[4] Sun et al., Microbiology 2002, 148, 361.
[5] Jian et al., Antonie Van Leeuwenhoek 2006, 90, 29.
[6] Kieser et al., The John Innes Foundation 2000, Norwich, England.
[7] Wohlleben et al., Acta Microbiol. Immunol. Hung. 1994, 41, 381.

Accession Numbers.

The nucleotide sequence of the pactamycin biosynthetic gene cluster and flanking regions from S. pactum ATCC 27456 has been deposited in GenBank accession number FJ392609 which is incorporated herein by reference in its entirety.

General DNA Manipulations:

Genomic DNA of S. pactum ATCC 27456 was prepared by standard protocol (Kieser et al., The John Innes Foundation, 2000, Norwich, England) or using the DNeasy Tissue Kit (QIAGEN). DNA fragments were recovered from an agarose gel by using the QIAquick Gel Extraction Kit (QIAGEN). Restriction endonucleases were purchased from Invitrogen or Promega. Preparation of plasmid DNA was done by using a QIAprep Spin Miniprep Kit (QIAGEN). All other DNA manipulations were performed according to standard protocols (Kieser et al., Sambrook et al., Molecular Cloning: A Laboratory Manual, 3^{rd} ed., 2001, Cold Spring Harbor Laboratory Press, NY). PCR was performed in 30 cycles by using a Mastercycler gradient thermocycler (Eppendorf) and Platinum Taq DNA polymerase (Invitrogen) or Platinum Pfx DNA polymerase (Invitrogen). Oligodeoxyribonucleotides for PCR primers were synthesized by Sigma-Genosys, and are shown in Table 6. The nucleotide sequences of the gene fragments were determined. ORFs were analyzed by FramePlot analysis (Ishikawa et al., FEMS Microbiol. Lett., 1999, 174:251) and BLAST programs (Altschul et al., J. Mol. Biol., 1990, 215:403).

TABLE 6

| Primer | Sequence^{(a)} | SEQ ID NO: |
|---|---|---|
| Apr-Fw/SphI | 5′-ACATGCATGCAATGGGTTCATGTGCAGCTC-3′ | 47 |
| Apr-Rv/SphI | 5′-ACATGCATGCAGCATATCATCAGCGAGCTG-3′ | 48 |
| CetM-F | 5′-GAAGATCTGCATATGAGCGGCCCTGGTTACCT-3′ | 49 |
| CetM-R | 5′-GGAATTCTCATTTCCTCGCAACCACTTCG-3′ | 50 |
| ptmA-F | 5′-TCCCGCTCGCTGTTCCGGTACTA-3′ | 51 |
| ptmA-R | 5′-TGTCCACGTCCTCGTCGGTCAT-3′ | 52 |
| ptmC-F1-HindIII | 5′-CCCAAGCTTTCCTTGAGGTGCAGCGCG-3′ | 53 |
| ptmC-R1-EcoRI | 5′-CCGGAATTCGTCGCGATGTGCGAACAT-3′ | 54 |
| ptmC-F2-EcoRI | 5′-CCGGAATTCCGTCTGCGGGAGCTGAAG-3′ | 55 |
| ptmC-R2-XbaI | 5′-TGCTCTAGAAGCTCCTCGACGAAGAAC-3′ | 56 |
| ptmC-P-1 | 5′-GTTGGCGACCGAGTTGAGC-3′ | 57 |
| ptmC-P-2 | 5′-GAACCCGTAGTAGTGCGTGGC-3′ | 58 |
| ptmJ-F1-HindIII | 5′-CCCAAGCTTGACTTCGACTACGTCCTC-3′ | 59 |
| ptmJ-R1-EcoRI | 5′-CCGGAATTCGACCACGCTCACCGAGAT-3′ | 60 |
| ptmJ-F2-EcoRI | 5′-CCGGAATTCGACGTCGGCACCTTCCTCGC-3′ | 61 |
| ptmJ-R2-XbaI | 5′-TGCTCTAGAGAGGCCGCACAGCAGCCATT-3′ | 62 |
| ptmJ-P-1 | 5′-CAACACCGAGTACCACGACAAGC-3′ | 63 |
| ptmJ-P-2 | 5′-GTCGCCGTCCCACTCCGGTTC-3′ | 64 |
| ptmQ-F1 | 5′-GAGGATCCGCATATGCCGGAGGGTACGGCCGG-3′ | 65 |
| ptmQ-S-R | 5′-ACCATGGGCAGCATGCCGCCCGCGCCGGCCA-3′ | 66 |
| ptmQ-S-F | 5′-GGAATTCGCGGCATGCTGCTGGTGGGGCTCTC-3′ | 67 |
| ptmQ-N-R | 5′-CCAGGGGAAGGCCATGGTCGGTACG-3′ | 68 |
| ptmQ-N-F | 5′-CGTACCGACCATGGCCTTCCCCTGG-3′ | 69 |
| ptmQ-R1 | 5′-GGAATTCTCAGGGGCGGGACGTGGTGACCA-3′ | 70 |
| ptmS-F | 5′-ATCAGCGATGTGTTCGCGATCATC-3′ | 71 |
| ptmS-R | 5′-ACGTTGATGCAGGTGGACAGCC-3′ | 72 |
| RifB-KS-F | 5′-GAGCCCGTCGCGATCGTC-3′ | 73 |
| RifB-KS-R | 5′-CGCTTCTTCGAGGATCATGT-3′ | 74 |

^{(a)}nucleotides in bold refer to restriction sites

Cloning and Sequencing of the ptm Gene Cluster:

Fosmid library preparation of S. pactum genomic DNA was carried out according to the protocols of CopyControl™ Fosmid Library Production Kit (EPICENTRE) by following the manufacturer's instructions. Three thousand fosmid colonies were obtained and used for screening of ptm gene cluster using heterologous probes as described below.

The ketosynthase (KS) domain in the rifB gene was amplified by PCR using Amycolatopsis mediterranei S699 genomic DNA as template and primers RifB-KS-F and RifB-KS-R (see Table 6). Plasmid DNA from the fosmid library was transferred onto Hybond™-N+ nylon membrane and Southern blot hybridization with the rifB KS domain probe was performed using digoxigenin (DIG)-labeling (DIG High Prime DNA Labeling and Detection Starter Kit II, Roche Applied Science) to give 44 positive fosmid colonies. The aminotransferase gene cetM from the cetoniacytone gene cluster was amplified from Actinomyces sp. Lu9419 genomic DNA using primers CetM-F and CetM-R (Table 6) and used to probe the 44 positive fosmid clones. The fosmid DNA was digested with ApaI, electrophoretically separated on an agarose gel, and transferred onto a Hybond™-N+ nylon membrane. Hybridization with the cetM probe gave 10 fosmid clones that were predicted to have both PKS and aminotransferase homologous sequences. In addition, the C-methyltransferase gene cloU from the clorobiocin biosynthetic gene cluster was also used to screen the 44 PKS-positive fosmids. Only one clone (fosmid TIP3) was positive for all three heterologous probes and was further analyzed for its involvement in pactamycin biosynthesis. Further screening to identify overlapping clones was carried out using PCR methods. Two homologous primer pairs were designed based on the aminotransferase gene (ptmA-F, ptmA-R) and the acyl-CoA synthetase gene (ptmS-F, ptmS-R), which are present on the left and right hand sides of TIP3, respectively, and were used to screen the fosmid library. DNA samples from the fosmid library were used as templates in the PCR screening. Three overlapping clones of TIP3 (5A7, 28F2, 24H11) were identified and the complete sequence of the pactamycin gene cluster was obtained using a combination of primer walking and pyrosequencing technologies carried out by Macrogen Inc.

Construction of ptmC and ptmJ Knock-Out Plasmids:

A 1 kb aac(3)IV gene along with multiple cloning site (MCS) of pTMN002 was introduced into the BamHI site of pHZ1358 (Sun et al., Microbiol, 2002, 148:361) to generate an apramycin resistant E. coli and Streptomyces shuttle vector pTMW018. For the construction of ptmC knock-out plasmid, two ˜1 kb PCR fragments upstream and downstream of the ptmC gene were generated using primers ptmC-F1-HindIII/ptmC-R1-EcoRI and ptmC-F2-EcoRI/ptmC-R2-XbaI (see Table 6), respectively, and separately ligated into HindIII/EcoRI and EcoRI/XbaI sites of pBluescript II SK(−) vector. The fusion product (2 kb) was subsequently excised with HindIII and XbaI and end-filled with Klenow and cloned into pTMW018 (after digested with BamHI and Klenow filled) to create pTMW028, which was introduced into S. pactum by conjugation using the E. coli donor strain ET12567 (pUZ8002). Apramycin resistant strains representing single crossover mutants were obtained and subsequently grown on nonselective mannitol-soy flour agar containing magnesium sulfate (10 mM) (MS—Mg) to allow the formation of double crossover recombinants. Apramycin sensitive colonies were counterselected by replica plating onto MS—Mg agar with and without apramycin (50 μg/mL). The resulting double-crossover candidate strains were confirmed by PCR amplification with external primers (ptmC-P-1 and ptmC-P-2, Table 6) flanking the targeted ptmC gene. The resulting PCR fragment from putative double crossover mutants was subcloned into pGEM-T Easy vector and sequenced to confirm that part of ptmC has been removed from the genomic DNA. A similar approach was used for the construction of ptmJ knock-out plasmid, except that the PCR fragments upstream and downstream of the ptmJ gene were generated using primers ptmJ-F1-HindIII/ptmJ-R1-EcoRI and ptmJ-F2-EcoRI/ptmJ-R2-XbaI, respectively.

Heterologous Expression of the 6MSA Synthase Gene:

Three fragments of the 6MSA synthase gene (ptmQ) were amplified by PCR with Taq DNA polymerase Hi-Fi (Invitrogen) using the fosmid clone TIP3 as template and primers [for ptmQfr1 (fragment 1), ptmQ-F1 and ptmQ-S-R; for ptmQfr2 (fragment 2), ptmQ-S-F and ptmQ-N-R; for ptmQfr3 (fragment 3), ptmQ-N-F and ptmQ-R1]. The three PCR products (ptmQ fr1-3) were digested with BamHI/NcoI, NcoI/EcoRI, and NcoI/EcoRI, respectively, and individually ligated into pJTU780 (digested by corresponding restriction enzymes) to generate pTMT001, pTMT002 and pTMT003. A 1.3 kb-SphI/NcoI-digested ptmQfr2 was ligated with SphI/NcoI-digested pTMT001 to obtain pTMT004. In order to obtain complete sequence of ptmQ, 2.1 Kb-EcoRI/NcoI-digested ptmQfr3 was then ligated with EcoRI/NcoI-digested pTMT004. The resulting plasmid, pTMT005 was then linearized by HindIII digestion and ligated with pGM9 vector. The product (pTMN102) was introduced into S. lividans T7 by a standard protoplast transformation method (Kieser et al., The John Innes Foundation 2000, Norwich, England). The transformants were grown in R2YE medium (25 mL), supplemented with kanamycin (50 μg/mL) and thiostrepton (7.5 μg/mL) (inducer), at 30° C. for 5 days. The culture supernatants were acidified to pH 2, extracted with ethyl acetate and dried. The samples were dissolved in acetonitrile before being subjected to LC-MS analysis. HPLC was carried out on an octadecyl silica gel column (Cosmosil 5C₁₈ AR-II 3.0×250 mm, Nacalai Tesque), flow rate 0.50 mL/min, solvent A (H₂O+0.1% formic acid) and solvent B (acetonitrile+0.1% formic acid). The following solvent gradient program was used: 0-5 min, % B=10; 5-25 min, % B=10-40; 25-30 min, % B=40-90; and 30-35 min, % B=10. Peaks eluting from the column were monitored at 254 nm. The molecular weight of each compound was determined by electrospray mass spectrometry (ThermoFinnigan). For GC-MS analysis, samples (10 mL) were evaporated to dryness, treated with SIGMA SIL-A for 30 minutes at 50° C. and dried under a gentle stream of argon. The derivatized products were resuspended in hexanes and analyzed on a Hewlett Packard 5890 Series II/Hewlett Packard 5971 Series GC/MS.

Synthesis of 6MSA:

A solution of 2-amino-6-methylbenzoic acid (3.0 g, 19.9 mmol) in 80% HCOOH was added to an aqueous solution (20 mL) of NaNO₂ (1.5 g, 21.9 mmol, 1.1 eq). The mixture was stirred at 0° C. for 1 hour, diluted with H₂O (120 mL), and warmed to 40° C. for 1 hour. The product was extracted with CHCl₃, washed with brine, and dried over Na₂SO₄, and the organic solvent was evaporated in vacuo. The residue was then dissolved in MeOH (10 mL) and passed through a charcoal column to remove the diazonium pigment. The eluent containing the product was dried in vacuo and the crude product was dissolved in MeOH/H₂O (10 mL, 1:1) and recrystallized. The crystals were filtered and dried to obtain pure 6MSA (1.9 g).

Construction of ptmQ Knock-Out Plasmid:

A 1 kb DNA fragment containing the Apr^{R} gene was cloned by PCR method using Apr-Fw/SphI and Apr-Rv/SphI as primers and pOJ446 as template. These primers were designed based on Apr^{R} gene sequence (accession no. X01385). Standard PCR conditions specified for Platinum Tag DNA polymerase were used. DNA fragments were digested with SphI and subcloned into SphI-digested pTMT005 vector. The product pTMN101 was digested with SalI and NcoI. The 3.4 kb SalI/NcoI digested ptmQ-Apr^{R} fragment was treated with Klenow fragment (M220A, Promega) and subsequently cloned into pHZ1358 vector (digested with BamHI and treated with Klenow fragment) to give pTMN001. The knock-out plasmid pTMN001 was transferred into E. coli ET12567 containing pUZ8002 and introduced into S. pactum by conjugation on MS plates. The plates were incubated at 30° C. for 16 hours before being treated with nalidixic acid (20 μg/mL) and apramycin (40 μg/mL), followed by incubation at 30° C. Apr^{R}/Thio^{S} colonies were counter-selected from initial Apr^{R}/Thio^{R} exconjugants after one round of nonselective growth. Three mutants were confirmed by step-up PCR and Southern hybridization.

Analysis of Metabolites from ptmC, ptmJ, and ptmQ Mutants:

S. pactum mutant strains were grown on BTT agar [glucose (1%), yeast extract (0.1%), beef extract (0.1%), casein hydrolysate (0.2%), agar (1.5%), pH 7.4] supplemented with appropriate antibiotics [apramycin (50 μg/mL) for the ptmQ mutant] at 30° C. for 3 days. Single colonies were used to inoculate the BTT seed cultures and incubated at 30° C. for 3 days. Production cultures were prepared in modified Bennet medium (Sakuda et al., J. Org. Chem., 2001, 66:3356) (25 mL) supplemented with appropriate antibiotics [apramycin (50 μg/mL) for the ptmQ mutant] and inoculated with seed cultures [10% (V/V)]. The production cultures were incubated at 30° C. for five days under vigorous shaking (200 rpm). The mycelia were centrifuged and the supernatants were extracted twice with equal volumes of ethyl acetate. The organic solvent was evaporated in vacuo and the residues were dissolved in MeOH and analyzed by reverse-phase HPLC or LC-MS.

Analysis of the metabolites of the ptmC and ptmJ mutants was carried out by reverse-phase HPLC on an octadecyl silica gel column (Cosmosil 5C₁₈AR-II 3.0×250 mm, Nacalai Tesque), flow rate 0.20 mL/min, solvent A (H₂O) and B (MeOH). The following solvent gradient program was used: 0-30 min, % B=50-100; 30-40 min, % B=100; and 42-55 min, % B=50. Peaks eluting from the column were monitored at 260 nm. Analysis of the ptmQ mutant metabolites was carried out using reverse-phase HPLC. The following solvent gradient program [solvent A (H₂O) and B (MeOH)] was used: 0-30 min, % B=50-100; 30-45 min, % B=100; and 50-60 min, % B=50. Peaks eluting from the column were monitored at 260 nm. The molecular weight of each compound was determined by electrospray mass spectrometry.

Purification and Characterization of De-6MSA-Pactamycin and de-6MSA-pactamycate:

The S. pactum mutants were cultivated in the production medium as described above. The supernatant, which was obtained from 10 L of culture, was loaded onto a H₂O-equilibrated Amberlite XAD-2 column (200 mL bed volume) and eluted with MeOH. The organic solvent was evaporated in vacuo and the remaining solution was subjected to Dowex 50W×2 column chromatography (H⁺ form, 50 mL bed volume). The column was washed with five volumes of H₂O and then eluted with NH₄OH (1 M, 100 mL). The eluted fraction was directly extracted with the same volume of ethyl acetate twice and the ethyl acetate extract was subsequently evaporated to dryness. The sample was subjected to silica gel column chromatography (CHCl₃-MeOH 100:1, 40:1, 20:1, 10:1 stepwise elution) to give fractions that contain de-6MSA-pactamycin (28) (the 20:1 fraction) and de-6MSA-pactamycate (30) (the 10:1 fraction). The 20:1 fraction was further subjected to preparative thin-layer chromatography (silica gel, n-BuOH—AcOH—H₂O 10:1:1) to yield pure de-6MSA-pactamycin (28, 2.1 mg). The 10:1 fraction was further subjected to silica gel column chromatography (CHCl₃-MeOH 10:1) to give pure de-6MSA-pactamycate (30, 25.2 mg).

28: White powder, [α]_{D}²²+2.1 (c 0.23, MeOH), HR-ESIMS m/z 425.2386 [M+H]⁺, calculated for C₂₀H₃₃N₄O₆ m/z 425.2400. ¹H and ¹³C NMR: as given in Table 7.

30: White powder, [α]_{D}²² −14.8 (c 0.64, MeOH), HR-ESIMS m/z 380.1844 [M+H]⁺, calculated for C₁₈H₂₆N₃O₆ m/z 380.1822, ¹H and ¹³C NMR: as given in Table 7.

Pactamycin and pactamycate were isolated from culture broths of S. pactum using the same procedures developed for de-6MSA-pactamycin and de-6MSA-pactamycate, respectively.

TABLE 7
NMR data for de-6MSA-pactamycin, de-6MSA-pactamycate, and pactamycate

| | de-6MSA-pactamycin | | de-6MSA-pactamycate | | pactamycate |
|---|---|---|---|---|---|
| position | ¹H (δ, ppm)* | ¹³C (δ, ppm)** | ¹H (δ, ppm)* | ¹³C (δ, ppm)** | ¹H (δ, ppm)* |
| 1 | | 72.8 | | 72.6 | |
| 2 | 3.08, d, 1H, | 62.3 | 3.53, d, 1H, | 60.7 | 3.60, d, 1H, |
| | J = 2.5 Hz | | J = 12 Hz | | J = 12 Hz |
| 3 | 3.78, d, 1H, | 69.3 | 3.70, d, 1H, | 71.1 | 3.67, d, 1H, |
| | J = 2.5 Hz | | J = 12 Hz | | J = 12 Hz |
| 4 | | 85.6 | | 83.0 | |
| 5 | | 89.5 | | 84.1 | |
| 6 | 1.47, s, 3H | 21.9 | 1.38, s, 3H | 17.6 | 1.38, s, 3H |
| 7 | 4.13, q, 1H, | 74.2 | 4.84, q, 1H, | 78.5 | 4.84, q, 1H, |
| | J = 6.5 Hz | | J = 8 Hz | | J = 8 Hz |
| 8 | 1.09, d, 3H, | 18.8 | 1.57, d, 3H, | 17.2 | 1.58, d, 3H, |
| | J = 6.5 Hz | | J = 6.5 Hz | | J = 6.5 Hz |
| 9 | 3.73, d, 1H, | 64.0 | 3.59, d, 1H, | 63.7 | 4.45, d, 1H, |
| | J = 11.5 Hz | | J = 11.5 Hz | | J = 11.5 Hz |
| | 3.96, d, 1H, | | 3.94, d, 1H, | | 4.64, d, 1H, |
| | J = 11.5 Hz | | J = 11.5 Hz | | J = 11.5 Hz |
| 10 | | 161.2 | | 161.1 | |
| 11 | 3.02, s, 6H | 37.2 | — | — | — |
| 12 | | | — | — | — |
| 1′ | — | — | — | — | — |
| 2′ | — | — | — | — | — |
| 3′ | — | — | — | — | Aromatic |
| 4′ | — | — | — | — | (3′, 4′, 5′), |
| 5′ | — | — | — | — | 7.0-7.4, m, 3H |
| 6′ | — | — | — | — | — |
| 7′ | — | — | — | — | 2.24, s, 3H |
| 1″ | — | 139.5 | — | 139.1 | — |
| 2″ | Aromatic | 113.3 | Aromatic | 113.2 | Aromatic |
| 3″ | (2″, 4″, 5″, 6″), | 149.7 | (2″, 4″, 5″, 6″), | 150.8 | (2″, 4″, 5″, 6″), |
| 4″ | 7.0-7.3, m, 4H | 118.5 | 7.0-7.4, m, 4H | 117.9 | 6.6-7.4, m, 7H |
| 5″ | | 130.7 | | 130.2 | |
| 6″ | | 119.6 | | 119.2 | |
| 7″ | — | 201.7 | — | 201.6 | — |
| 8″ | 2.60, s, 3H | 27.0 | 2.58, s, 3H | 26.9 | 2.44, s, 3H |

*Bruker, 300 MHz, CD₃OD;
**Bruker, 75.5 MHz, CD₃OD.

Identification and Sequencing of the Pactamycin Gene Cluster:

To obtain the biosynthetic gene cluster of pactamycin in S. pactum ATCC 27456, a genomic library was constructed using the Copy Control Fosmid system (EPICENTRE). The library was screened using a number of heterologous probes including the ketosynthase domain of rifB (a polyketide synthase (PKS) module from the rifamycin gene cluster) (August et al., Chem. Biol., 1998, 5:69), the cetM (aminotransferase) gene from the cetoniacytone A biosynthetic gene cluster (Wu et al., Chembiochem., 2009, 10:304), and the cloU (C-methyltransferase) gene from the clorobiocin biosynthetic gene cluster (Freitag et al., Microbiol., 2006, 152:2433). Initial screening using the PKS probe (rifB) resulted in 44 positive clones. Further screening of these 44 PKS-positive clones using either cetM or cloU resulted in ten and six positives, respectively. Among them, only one clone (TIP3) was positive with all three different probes. Further screening using PCR methods resulted in the identification of three additional fosmid clones that house DNA fragments containing flanking regions upstream and downstream of that in TIP3.

An 86 kb region of DNA containing the pactamycin gene cluster was sequenced using a combination of pyrosequencing, shotgun sequencing, and primer walking methodologies. Analysis of the sequence using BLAST search indicated the presence of 52 ORFs, from which 26 ORFs (ptmA-ptmZ) are considered to be the core cluster directly involved in the biosynthesis of pactamycin. These studies are described in U.S. patent application Ser. No. 12/596,429 filed on Oct. 16, 2009 (and published as US 2010/0210837 A1), which is the U.S. National Stage of International Application No. PCT/US2008/060876, filed Apr. 18, 2008 (and published as WO 2008/131258), which in turn claims the benefit of the earlier filing date of U.S. Provisional Application No. 60/912,824, filed Apr. 19, 2007; each of these applications is hereby incorporated by reference in its entirety. A number of additional genes upstream and downstream of the core cluster may be involved in the transcriptional regulation of the pathway and/or in resistance mechanisms. These include orf11 (encodes protein that shares high homology with the extracytoplasmic function (ECF) subfamily of RNA polymerase sigma factors), orf14 and orf15 (encode proteins that have low identity to translation initiation factor IF-2 from Frankia alni ACN4a and Streptomyces avermitilis MA-4680, respectively), orf9, orf16, and orf19 (encode proteins that are highly related to the family of ATP-dependent (DEAD-box) RNA helicases), orf18 (encodes protein that shares high homology with nourseothricin acetyltransferase from Streptomyces noursei), and orf23 (encodes protein that is highly similar to the tRNA methyltransferase from S. avermitilis). The pactamycin gene cluster reported by Kudo et al. consists of orf1, pctA-pctX, and orf2, -3, and -4 (Kudo et al., J. Antibiot., 2007, 60:492), which are identical with orf16, the ptm genes, and orf17.

Development of a Genetic System for Gene Inactivation Studies with S. pactum:

Gene inactivation studies represent the gold standard for confirming the involvement of putative gene clusters in the biosynthesis of secondary metabolites and have not been previously developed in the pactamycin producer, due to difficulties working with this strain.

A modified pHZ1358 vector, which has previously been used for generating deletion mutations within the validamycin biosynthetic gene cluster backbone, was used for the conjugation experiments (Bai et al., Chem. Biol., 2006, 13:387; Minagawa et al., Chembiochem., 2007, 8:632). This vector contains a thiostrepton resistance (Thio^{R}) gene and an OriT transfer element required for conjugation. Since S. pactum has much greater sensitivity to apramycin than to thiostrepton, a 1 kb aac(3)IV apramycin resistance gene along with multiple cloning site isolated from pTMN002 (Table 5) was introduced into the BamHI site of pHZ1358 to generate pTMW018. pTMW018 was used in gene inactivation experiments with S. pactum as described below.

Inactivation of the Fe—S Radical SAM Oxidoreductase (ptmC) and the Glycosyltransferase (ptmJ) Genes:

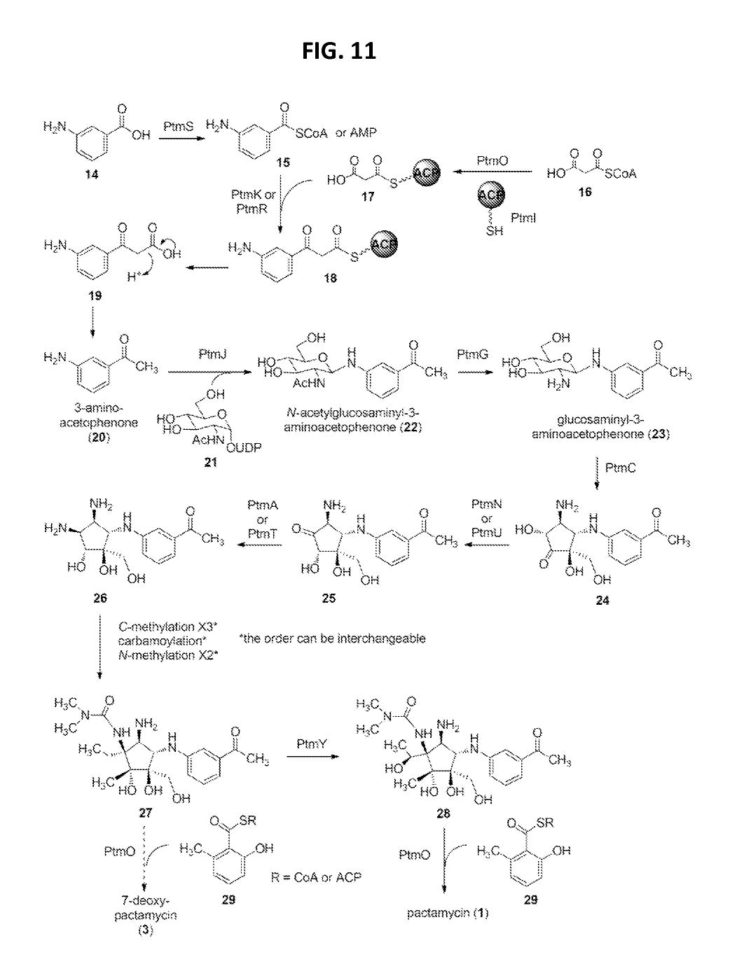

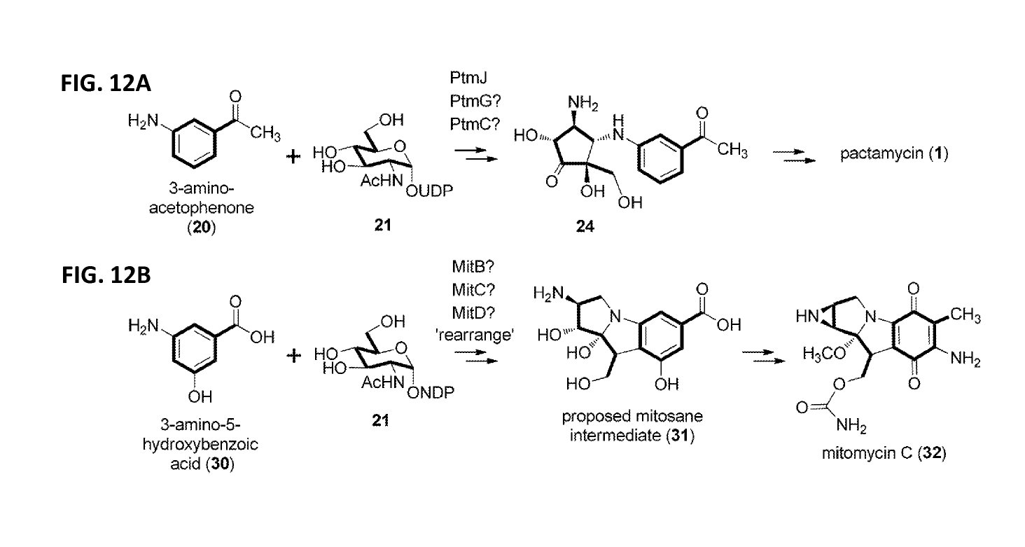

In silico analysis of PtmC has revealed that this enzyme lacks a B12-like binding motif and more closely resembles the subfamily of radical SAM enzymes involved in redox chemistry. Based on BLAST similarity with the mitomycin biosynthetic genes and preliminary studies et al., it was predicted that PtmC, PtmJ (putative glycosyltransferase), and PtmG (putative deacetylase) are involved in the formation of the cyclopentitol core 24 (FIG. 11), and that this process is similar to the formation of the mitosane core structure during mitomycin (32) biosynthesis (FIG. 12) (Mao, Chem. Biol., 1999, 6:251). The radical SAM enzyme MitD, the glycosyltransferase MitB, and the putative N-deacetylase MitC from the mitomycin biosynthetic gene cluster are close homologues of PtmC, PtmJ, and PtmG, respectively. During mitomycin biosynthesis, D-glucosamine or its derivative is assembled into the mitosane core structure via condensation with 3-amino-5-hydroxybenzoic acid (AHBA). Although the details of this reaction mechanism have not been elucidated, it was believed that MitB would mediate the condensation reaction between the two units followed by an additional mechanism to complete the C—C bond formation (Mao et al., J. Bacteriol., 1999, 181:2199). In the case of pactamycin biosynthesis, it has been demonstrated that the MitB homologue PtmJ (PctL) is capable of coupling UDP-N-acetyl-α-D-glucosamine and 3-aminoacetophenone. Intermediate 22 would then need to undergo deacetylation, possibly by the N-deacetylase homologue PtmG followed by radical-mediated rearrangement by PtmC to form the cyclopentitol ring structure.

To confirm the involvement of ptmC and ptmJ in pactamycin biosynthesis, the genes were individually inactivated and the phenotypes were characterized by LC-MS. The targeted in-frame deletion of ptmC and ptmJ were achieved by cloning upstream and downstream flanking regions surrounding the functional domain of ptmC and ptmJ, respectively, into pTMW018, transferring the plasmids to S. pactum via conjugation and selecting for double crossover recombinants lacking part of the ptmC and ptmJ gene sequences. PtmC and ptmJ mutant strains (S. pactum MT-ptmC and MT-ptmJ) were confirmed by PCR amplification and DNA sequencing of the subcloned products. LC-MS analysis of the culture broths of the ptmC and ptmJ mutant strains revealed the lack of pactamycin biosynthesis in these strains, which directly confirmed the involvement of these genes in pactamycin biosynthesis.

Heterologous Expression of the 6-MSA Synthase Gene (ptmQ):

In order to further validate the cluster authenticity and develop a heterologous expression system, the putative polyketide synthase (PKS) gene, ptmQ, in S. lividans. Was cloned and heterologously expressed in S. lividans. PtmQ shares high similarity with the iterative type I PKS, Ch1B1, that is involved in the biosynthesis of 6-MSA in Streptomyces antibioticus (Jia et al., Chem. Biol. 2006, 13:575). The 5.5 kb putative 6-MSA synthase gene (ptmQ) was subcloned into pJTU780, which was derived from pRSET B (Invitrogen). The resulting plasmid (pTMT005) was then linearized by HindIII digestion and ligated with pGM9 vector (Wohllebel et al., Acta Microbiol. Immunol. Hung., 1994, 41:381). Plasmid pGM9 can replicate in S. lividans, but not in E. coli. The fusion of the entire pTMT005 plasmid into pGM9 resulted in a shuttle plasmid that can replicate in both E. coli and S. lividans. The product (pTMN102) was introduced into S. lividans T7 and the production of 6-MSA from the transformants was analyzed by LC-MS. A new peak corresponding to 6-MSA was detected in a sample prepared from the culture of transformant P2-3, which harbors the ptmQ gene, compared with the sample prepared from the culture of S. lividans harboring the empty vector pGM9. In order to verify this peak as 6-MSA, the samples were co-injected with an authentic 6-MSA standard, which was synthesized from 2-amino-6-methylbenzoic acid in the presence of formic acid and NaNO₂. The co-elution of the mutant product with the synthetic compound supported the identity of the new peak as 6-MSA. Further confirmation was achieved by GC-MS analyses after TMS derivatization of the samples.

Inactivation of ptmQ and Metabolic Analysis of ptmQ Mutants:

In addition to heterologous expression, the PKS gene (ptmQ) was targeted for gene disruption. Double crossover recombination was used to introduce an apramycin resistance gene into the SphI restriction site located in the ptmQ gene. Thus, the knock-out plasmid was prepared by inserting the 1 kb aac(3)IV gene into the SphI site (located in the ptmQ gene) of pTMT005 and the product was subsequently digested with SalI and NcoI to give a 3.4 kb DNA fragment harboring the aac(3)IV gene flanked by part of ptmQ sequence on each sides. The DNA fragment was then inserted into pHZ1358 vector and the resulting plasmid was transferred into S. pactum ATCC 27456 through conjugation. Genomic DNA from three resulting mutant strains (PKSM1/3, PKSM1/8, PKSM1/19) was analyzed by step-up PCR (Simpson et al., J. Biogeogr., 2005, 32:14) and Southern hybridization to confirm the insertion of the apramycin cassette into the ptmQ gene.

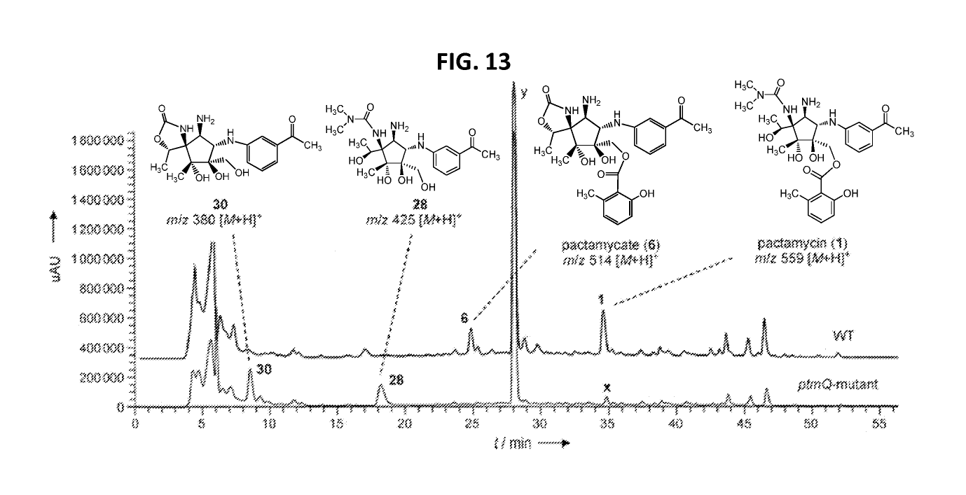

To investigate the effect of ptmQ inactivation on pactamycin biosynthesis, the wild-type and the ptmQ mutant strains of S. pactum were cultivated in modified Bennet medium (Sakuda et al., J. Org. Chem., 2001, 66:3356) and the metabolites were analyzed by LC-MS. As shown in FIG. 13, the ptmQ mutants were not able to produce pactamycin (1) and/or pactamycate (6), but instead produced two new metabolites, 28 (MW=424) and 30 (MW=379).

Structural Elucidation of de-6MSA-Pactamycin and de-6MSA-pactamycate:

The chemical structures of 28 and 30 were determined based on their mass spectrometric and NMR spectroscopic data (Table 7). The ¹H and ¹³C NMR spectra of 28 and 30 showed signals similar to those of the known compounds pactamycin and pactamycate, respectively, except that a number of signals corresponding to the protons and carbons of 6-MSA were missing in those of the new compounds. In particular, the methyl signals unique to the 7′-position of 6-MSA and a number of signals in the aromatic regions were missing in the NMR spectra of both 28 and 30. Furthermore, the H₂-9 signals of 28 (δ_{H} 3.73 and 3.96 ppm) and 30 (δ_{H} 3.59 and 3.94 ppm) appeared up-field of those of pactamycate (δ_{H} 4.45 and 4.64 ppm), indicating that the former compounds lack an ester bond at this position. The data indicate that 28 and 30 lack the 6-MSA moiety in their structures.

Pactamycate is a derivative of pactamycin, in which the C-7 hydroxyl attacks the neighboring 1,1-dimethylurea group to form a 2-oxazolidone ring, resulting in the loss of dimethylamino group. Compound 30 was predicted to be the 2-oxazolidone derivative of 28, as it has 45 atomic mass units less than 28, which corresponds to the loss of a nitrogen atom and two methyl groups. The ¹H NMR spectrum of 30 showed, among others, signals related to three methyl groups, as oppose to five in that of 28. Similarly, three methyl carbons (C-6, C-8, C-8″) were observed in the ¹³C NMR spectrum of 30, whereas five carbons (C-6, C-8, C-11, C-12, C-8″) were present in that of 28. Furthermore, the methyl signal at 37.2 ppm, which corresponds to C-11 and C-12, was observed only in the ¹³C NMR spectrum of 28, confirming the lack of N-methyl groups in 30. All together these data support the assignment of 28 and 30 as de-6MSA-pactamycin and de-6MSA-pactamycate, respectively.

The production of compounds 28 and 30 suggests that hydroxylation of the C-7 position occurs prior to the attachment of 6-MSA during pactamycin biosynthesis, and that compound 28 is a primary intermediate en route to pactamycin. The previous isolation of 7-deoxypactamycin suggested that the acyltransferase enzyme that is involved in the condensation of 6-MSA with the cyclopentitol moiety is rather flexible in terms of its substrate specificity. Thus, 7-deoxypactamycin is predicted to be an endpoint product that is produced as part of a minor alternative shunt pathway and that it is not modified further to yield pactamycin. This is in contradiction to the previous notion that 7-deoxypactamycin is a direct precursor in the pathway. The generation of the ptmQ mutant strains and the production of compounds 28 and 30 provide invaluable tools for generating a library of pactamycin analogs that would represent pharmaceutical leads from an untapped chemical class.

Antimalarial Activity Assay:

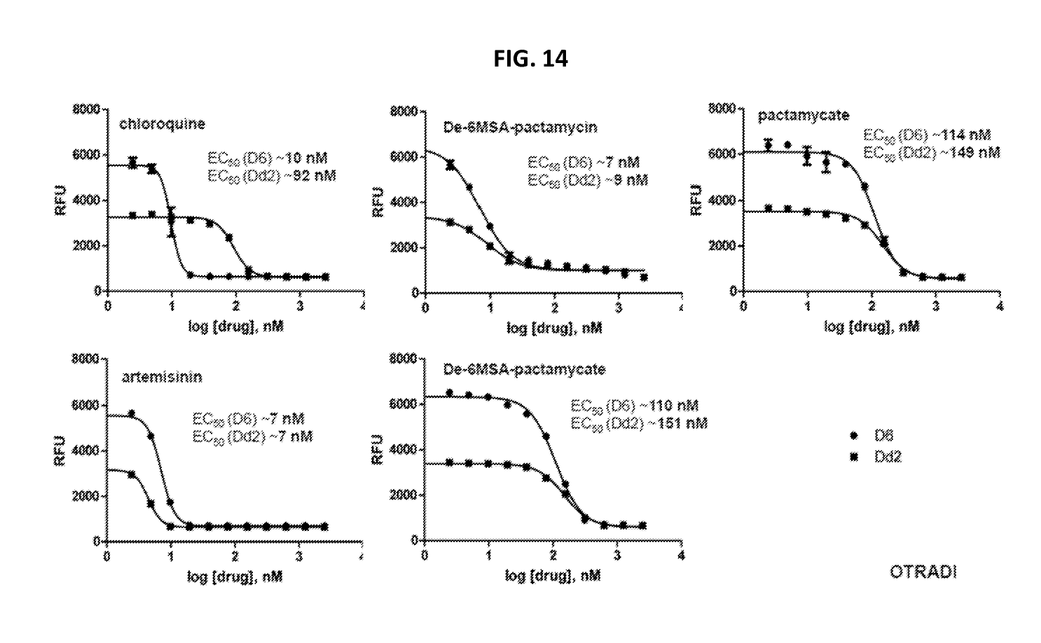

De-6MSA-pactamycin and de-6MSA-pactamycate also were evaluated for antimalarial activity (FIG. 14) against the D6 and Dd2 strains. De-6MSA-pactamycin had an EC₅₀ value of 7 nM against the D6 strain, compared to an EC₅₀ value of 10 nM for chloroquine. De-6MSA-pactamycin was 10 times more effective than chloroquine against the multi-drug resistant Dd2 strain with an EC₅₀ of 9 nM, and showed similar activity to artemisinin (EC₅₀ of 7 nM) against Dd2. De-6MSA-pactamycate exhibited similar activity to pactamycate against both strains.

Antibacterial Activity Assay:

Antibacterial activity of pactamycin and its analogues was determined by an agar diffusion assay. E. coli, Pseudomonas aeruginosa, Mycobacterium smegmatis, and Staphylococcus aureus were streaked on nutrient agar (Difco) and grown overnight at 37° C. Colonies were transferred to nutrient broth and incubated at 37° C. for 24 hours. Turbidity of the inoculum was measured to a proper density at 600 nm (BioRad, SmartSpec 3000). For plate preparation, inoculum (500 μL) was mixed thoroughly with warm nutrient agar (50 mL) and poured to 25 mL square plates. The agar plates were allowed to solidify and dry for 30 minutes before assay. Sterile blank paper disks (Becton-Dickinson) were impregnated with pactamycin and its analogues (20 μL) at various concentrations and dried at room temperature. The disks were placed onto inoculated agar plates and incubated at 37° C. for 24 hours. In order to produce a contrast background of the inhibition zone, 0.25% MTT developing dye (1 mL) was added over the plates.

Dose response assays were performed at a 100-μL volume in 200 μL Eppendorf tubes. The compounds were diluted and added to the bacterial suspension at final concentrations of 0.01 (or 0.1)-100 μM. After incubation at 37° C. for 24 hours, turbidity of cultures was measured at 600 nm (Biorad, SmartSpec 3000). Each assay was done in triplicate.

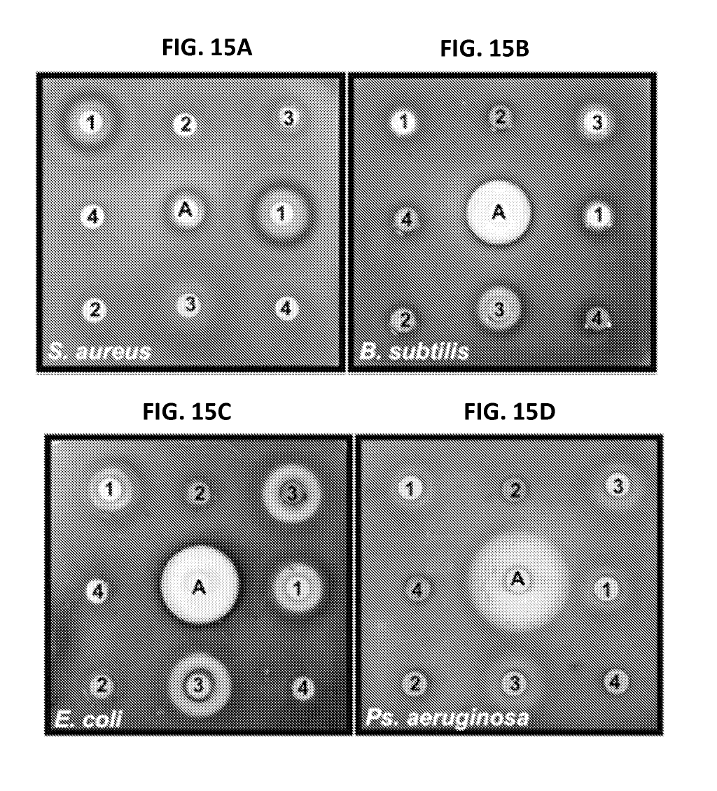

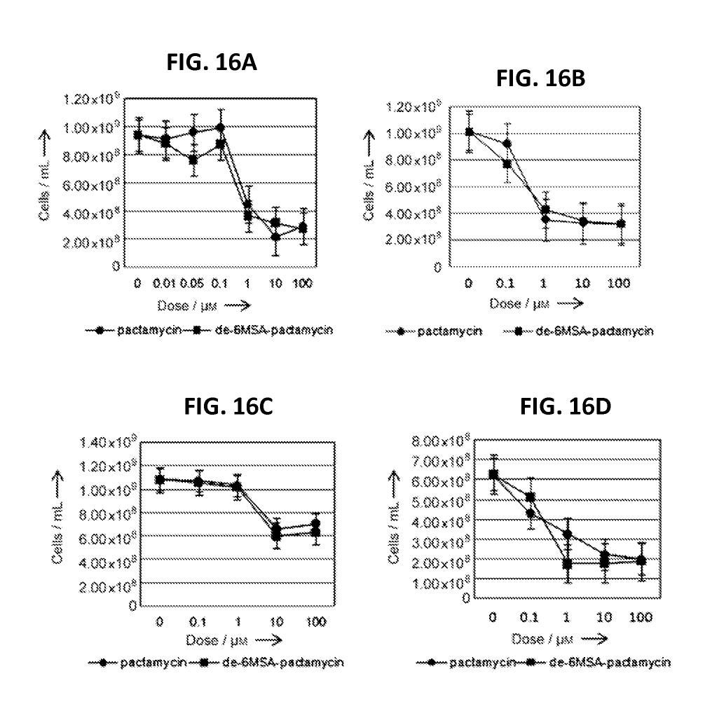

In this analysis pactamycate and de-6MSA-pactamycate showed no antibacterial activity, whereas de-6MSA-pactamycin and pactamycin inhibited the growth of Mycobacterium smegmatis, Staphylococcus aureus, Pseudomonas aeruginosa, and E. coli (FIGS. 15A-15D). To further evaluate and compare the in vitro efficacy of de-6MSA-pactamycin with that of pactamycin, dose-response studies were done using a broth microdilution method with 0.01 (or 0.1)-100 μM concentrations of pactamycin and de-6MSA-pactamycin (FIGS. 16A-16D). In all of the studies, de-6MSA-pactamycin showed equivalent biological activity with the parent molecule, pactamycin, indicating that the 6-MSA moiety is not essential for activity. Both pactamycin and de-6MSA-pactamycin were maximally active between 1 and 10 μM and effective against both Gram-positive and Gram-negative bacteria.

Cytotoxic Activity and Cell Viability:

The MTT assay was carried out as described by Mosmann (J. Immunol. Methods, 1983, 65:55). Briefly, human colon cancer HT-29 cells (ATCC) were seeded in 96-well plates at a density of 1.0×10⁴ cells per well. The growth medium consisted of phenol red-free RPMI 1640 (Invitrogen, Carlsbad, Calif.) containing 10% fetal bovine serum (Invitrogen, Carlsbad, Calif.), glutamine (2 mM), penicillin (100 units/mL) and streptomycin (100 μg/mL). After a 24-hour incubation at 37° C. in a humidified atmosphere of 5% CO₂, the medium was removed and replaced with fresh RPMI 1640 medium (with or without 10% FBS and no penicillin or streptomycin). Increasing concentrations of pactamycin, pactamycate, de-6MSA pactamycin, or de-6MSA-pactamycate dissolved in DMSO were added to cells at a volume of 0.2 mL/well. DMSO was added to control media not to exceed a final concentration of 1%. Following a 24-hour incubation, the medium was aspirated from each well and replaced with fresh RPMI medium with 10% FBS and MTT (0.5 mg/mL). The cells were incubated for 3 hours at 37° C. and then the MTT-containing media was removed and replaced with isopropanol containing 0.04 N HCl (200 μL/well). The plate was shaken for 10 minutes before reading the absorbance at 570 nm using a microplate reader (SpectraMax 250, Molecular Devices, Sunnyvale, Calif., USA). The viability of treated cells was expressed as the percentage of control cells. Cells treated with 10% TCA in H₂O for 24 hours were used as positive controls (100% lethality). The studies were replicated three times.

Growth inhibition of HT-29 cells induced by the test compounds was also evaluated by the SRB assay, which is based on measuring total biomass by staining the cellular proteins with sulforhodamine B (Skehan et al., J. Natl. Cancer Inst., 1990, 82:1107). After treating the HT-29 cells with the compounds for 24 hours, the adherent cells grown in 96-well plates were fixed with 10% trichloroacetic acid and incubated for 1 hour at 4° C. The plates were washed five times with tap water, dried, and stained with SRB solution (0.4% wt/vol in 1% acetic acid) for 30 minutes at room temperature. The plates were again washed and dried before adding 10 mM Tris base to solubilize the bound stain. Optical densities (OD) were read at 564 nm in SpectraMax 250 and cell growth of treated cells was expressed as the percentage of control cells, ODtest/ODcontrol×100%.

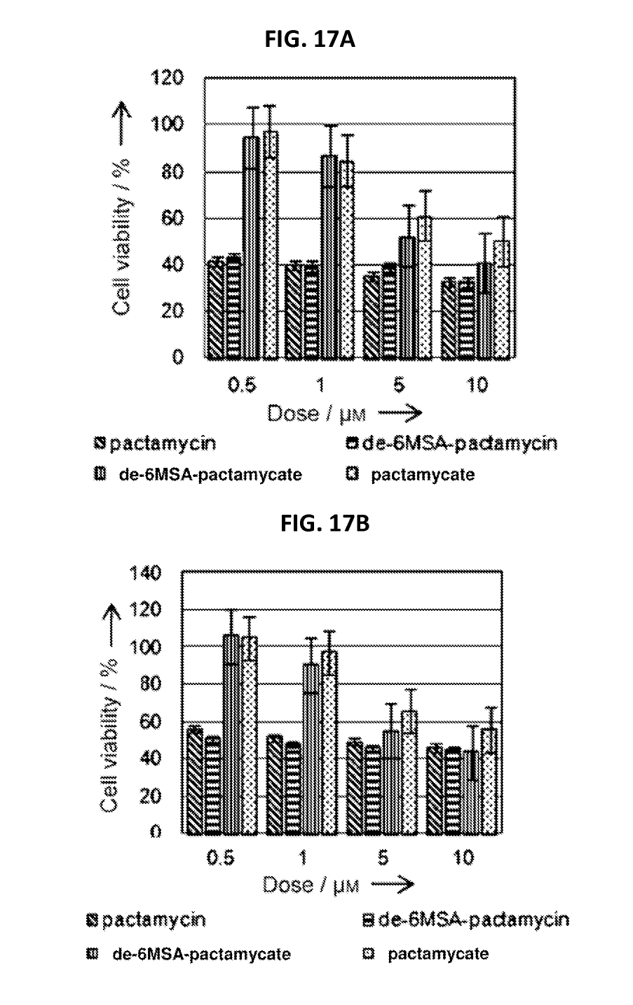

Cytotoxic activity of de-6MSA-pactamycin and de-6MSA-pactamycate was evaluated and compared with the parent compounds in sulforhodamine (SRB)- and MTT-based assays using HT-29 human colon carcinoma cells. Results from these studies show that both de-6MSA-pactamycin and de-6MSA-pactamycate retained equivalent cytotoxicity profiles with the respective parent compounds (FIGS. 17A-17B). Inclusion of fetal bovine serum (FBS) in the growth medium of the HT-29 cells did not have any effect on cell viability following exposure to pactamycin or its analogues.

Taken together, the cytotoxicity data combined with the antibiotic bioactivity profiles and other previous reports on the biological activity of pactamycin, the structure-activity relationship (SAR) of pactamycin is more readily understood. The antibacterial activity of pactamycin has previously been shown to be dependent on its ability to bind with the 30S ribosomal subunit and disrupt the ability of certain mRNA-tRNA₂ complex to translocate from the A and P sites to the P and E sites, respectively (Dinos et al., Mol. Cell, 2004, 13:113). The loss of antibacterial activity and reduced cytotoxicity profile of pactamycate and de-6MSA-pactamycate indicate that the 1,1-dimethylurea functionality plays a role in activity. In fact, the crystal structure of pactamycin bound to the 30S ribosomal subunit shows that pactamycin mimics a dinucleotide RNA structure and binds in the E-site cleft of the ribosome. Hydrogen bonding is predicted to occur with both the carbonyl oxygen of the 1,1-dimethylurea moiety and the primary amine at the C-2 position on the cyclopentitol ring structure. Both of these moieties are involved in the function of pactamycin. Loss of the hydroxyl functionality at the C-7 position of the cyclopentitol ring or the 6-MSA functionality appears to have no impact on the biological activity of the compounds, indicating that these positions are more flexible and not directly involved in ribosomal binding.

### Example 3

### Methods to Treat Colorectal Cancer

This example describes a particular representative method that can be used to treat colorectal cancer in humans by administration of one or more of the disclosed pactamycin analogs. One skilled in the art will appreciate that variations from the described method can be made without substantially affecting the treatment.

Based upon the teaching disclosed herein, colorectal cancer can be treated by administering an effective amount of a disclosed pactamycin analog, thereby reducing or eliminating a sign or symptom associated with colorectal cancer, such as tumor growth.

Briefly, the method can include screening subjects to determine if they have colorectal cancer. Subjects having colorectal cancer are selected. In one example, a clinical trial would include half (or some other proportion) of the subjects following the established protocol for treatment of colorectal cancer (such as a normal chemotherapy/radiotherapy/surgery regimen). The other portion of the subjects would follow the established protocol for treatment of the colorectal cancer (such as a normal chemotherapy/radiotherapy/surgery regimen) in combination with administration of the therapeutic compositions described above. In some examples, the tumor is surgically excised (in whole or part) prior to treatment with the therapeutic compositions. In another example, a clinical trial would include half of the subjects following the established protocol for treatment of colorectal cancer (such as a normal chemotherapy/radiotherapy/surgery regimen). The other half would follow administration of a therapeutic compositions described herein. In some examples, the tumor is surgically excised (in whole or part) prior to treatment with the therapeutic composition.

### Screening Subjects

In some examples, subjects are first screened to determine if they have colorectal cancer. Examples of methods that can be used to screening for colorectal cancer include diagnostic imaging (such as x-rays, CT scans, MRIs, ultrasound, fiber optic examination, and laparoscopic examination), tissue biopsy to detect a molecule indicative of a tumor (such as a colorectal tumor marker), and analyzing serum blood levels for a molecule indicative of a tumor (such as a colorectal tumor marker). If blood or a fraction thereof (such as serum) is used, 1-100 μl of blood is collected. Serum can either be used directly or fractionated using filter cut-offs to remove high molecular weight proteins. If desired, the serum can be frozen and thawed before use. If a tissue biopsy sample is used, 1-100 μg of tissue is obtained, for example using a fine needle aspirate. The biological sample (e.g., tissue biopsy or serum) is analyzed to determine if one or more signs of colorectal cancer are present, such as expression of one or more biomarkers known to be indicative of colorectal cancer.

Detection of one or more signs of colorectal cancer is indicative that the subject has colorectal cancer and is a candidate for receiving the therapeutic compositions disclosed herein. However, such pre-screening is not required prior to administration of the therapeutic compositions disclosed herein (such as those that include a disclosed pactamycin analog).

### Pre-Treatment of Subjects

In particular examples, the subject is treated prior to administration of a therapeutic composition that includes one or more disclosed pactamycin analogs. However, such pre-treatment is not always required, and a skilled clinician can determine whether it is. For example, the tumor can be surgically excised (in total or in part) prior to administration of the therapy. In addition, the subject can be treated with an established protocol for treatment of the particular tumor present (such as a normal chemotherapy/radiotherapy regimen).

### Administration of Therapeutic Compositions

Following subject selection, a therapeutic effective dose of the composition is administered to the subject, wherein the composition includes one or more pactamycin analogs, such as 7-demethyl-7-deoxypactalactam. Administration of the therapeutic compositions can be continued after chemotherapy and radiation therapy is stopped and can be taken long term (for example over a period of months or years).

### Assessment

Following the administration of one or more therapies, subjects having a malignant tumor (for example colorectal cancer) can be monitored for tumor improvement or change, such as regression or reduction in metastatic lesions, tumor growth or vascularization. In particular examples, subjects are analyzed one or more times, starting 7 days following treatment. Subjects can be monitored using any method known in the art. For example, diagnostic imaging can be used (such as x-rays, CT scans, MRIs, ultrasound, fiber optic examination, and laparoscopic examination), as well as analysis of biological samples from the subject (for example analysis of blood, tissue biopsy, or other biological samples), such as analysis of the type of cells present, or analysis for a particular tumor marker. In one example, assessment can be made using ultrasound, MRI, or CAT scans, or analysis of the type of cells contained in a tissue biopsy. It is also contemplated that subjects can be monitored for the response of their tumor(s) to therapy during therapeutic treatment by at least the aforementioned methods.

### Additional Treatments

In particular examples, if subjects are stable or have a minor, mixed or partial response to treatment, they can be re-treated after re-evaluation with the same schedule and preparation of agents that they previously received for the desired amount of time, such as up to a year of total therapy. A partial response is a reduction in size or growth of some tumors, but an increase in others.

### Example 4

### Methods of Treatment and Prophylaxis of a Pathogen Infection

This example provides representative methods that can be used to treat a subject having a pathogen infection, or to prevent or reduce the incidence of a future infection. Methods of treatment include methods that reduce one or more symptoms due to the infection in the subject, such as fever or increased white blood cell count. However, complete elimination of symptoms is not required. Treatment methods can also include reducing the presence of the pathogen, such as reducing viral titer in a subject. Prophylactic methods include reducing the incidence of a future pathogen infection, for example in a subject who is susceptible to infection by the pathogen (such as children, the elderly, and medical workers).

In particular examples, the method includes administering to the subject an effective amount of a disclosed pactamycin analog to a subject alone, or in combination with other agents, such as a pharmaceutical carrier, other therapeutic agents (such as anti-malaria compounds), or combinations thereof. In on example, the subject is a mammal, such as mice, non-human primates, and humans.

In particular examples, a subject susceptible to or suffering from an infection, wherein decreased infection by the pathogen is desired, is treated with a disclosed pactamycin analog. After the agent has produced an effect (a decreased level of pathogen infection is observed, or symptoms associated with infection decrease), for example after 24-48 hours, the subject can be monitored for diseases associated with the infection.

In particular examples, the subject is first screened to determine the type of pathogen infection present. If the pathogen is one that can be decreased by the disclosed therapies, the subject is then administered the therapy.

The treatments disclosed herein can also be used prophylactically, for example to inhibit or prevent infection by a pathogen. Such administration is indicated where the treatment is shown to have utility for treatment or prevention of the disorder. The prophylactic use is indicated in conditions known or suspected of progressing to disorders associated with a pathogen infection.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. I therefore claim as my invention all that comes within the scope and spirit of these claims.

1. A method of treating a parasitic infection in a subject, comprising:
selecting a subject for treatment that has, or is at risk for developing, a parasitic infection; and
administering to the subject an effective amount of a pharmaceutical composition comprising a compound other than pactamycin, 7-deoxypactamycin, or pactamycate, the compound having a general formula
where R¹ is H, lower aliphatic, amide, acyl, or aminoacyl;
R² is —C(O)NR⁸R⁹ where R⁸ and R⁹ independently are hydrogen or lower aliphatic, or R¹ and R² together form a cyclic structure;
R³ and R⁴ independently are hydrogen, hydroxyl, or lower aliphatic, or R² and R³ together form a cyclic structure;
R⁵ is hydrogen or acyl; and
R⁶ and R⁷ independently are hydrogen, hydroxyl, halogen, lower aliphatic, or amino, thereby treating the parasitic infection in the subject.

2. The method of claim 1, wherein R³ and R⁴ are hydrogen and the compound has the general formula

3. The method of claim 2, wherein R⁵ is —C(O)R¹⁰ wherein R¹⁰ is
wherein X is H, lower alkyl, —OR¹¹, halogen, —NO₂, or —NR¹²R¹³; Y is H, halogen or lower alkyl; and R¹¹, R¹² and R¹³ independently are H, lower alkyl or acyl.

4. The method of claim 1, wherein R¹ and R² together form a heterocyclic 5-membered ring.

5-6. (canceled)

7. The method of claim 1, wherein R² and R³ together form a heterocyclic 5-membered ring.

8. (canceled)

9. The method of claim 1, wherein R⁵ is a substituted benzoyl group.

10. (canceled)

11. The method of claim 1, wherein the compound has a formula selected from

12-13. (canceled)

14. A compound other than pactamycin, 7-deoxypactamycin, or pactamycate, the compound having a general formula
where R¹ is H, lower aliphatic, amide, acyl, or aminoacyl;
R² is —C(O)NR⁸R⁹ where R⁸ and R⁹ independently are hydrogen or lower aliphatic, or R¹ and R² together form a cyclic structure;
R³ and R⁴ independently are hydrogen, hydroxyl, or lower aliphatic, or R² and R³ together form a cyclic structure;
R⁵ is hydrogen or acyl;
R⁶ is hydrogen, hydroxyl, lower aliphatic, or amino; and
R⁷ is hydrogen, halogen, lower aliphatic, or amino,
wherein if R³, R⁸, and R⁹ are methyl, then at least one of R¹ or R⁴ is lower aliphatic or at least one of R⁶ and R⁷ is not hydrogen, wherein if R³ is alkyl or hydrogen and R⁴ is hydrogen or hydroxyl, then at least one of R¹, R⁵, R⁶, and R⁷ is not hydrogen, and wherein if R² and R³ together form a cyclic structure, then R⁴ is not methyl or at least one of R¹, R⁶, and R⁷ is not hydrogen.

15. The compound of claim 14, wherein R³ and R⁴ are hydrogen, and the compound has the general formula

16. The compound of claim 14, wherein R⁵ is —C(O)R¹⁰ wherein R¹⁰ is
wherein X is H, lower alkyl, —OR¹¹, halogen, —NO₂, or —NR¹²R¹³; Y is H, halogen or lower alkyl; and R¹¹, R¹² and R¹³ independently are H, lower alkyl or acyl.

17. The compound of claim 14, wherein R¹ and R² together form a heterocyclic 5-membered ring.

18. (canceled)

19. The compound of claim 14, wherein R² and R³ together form a heterocyclic 5-membered ring.

20. (canceled)

21. The compound of claim 14, wherein R⁵ is a substituted benzoyl group.

22. (canceled)

23. The compound of claim 14, having a formula selected from

24. A pharmaceutical composition, comprising a compound according to claim 14 and a pharmaceutically acceptable carrier.

25. A method of inhibiting a tumor in a subject, comprising:
selecting a subject for treatment that has, or is at risk for developing, a tumor;
administering to the subject an effective amount of the pharmaceutical composition of claim 24, thereby treating the tumor in the subject.

26. A method of treating an infection from a pathogen of interest in a subject, comprising:
selecting a subject for treatment that has, or is at risk for developing, an infection by a pathogen of interest;
administering to the subject an effective amount of the pharmaceutical composition of claim 24, thereby treating the infection from the pathogen of interest in the subject.

27. The method of claim 26, wherein the pathogen of interest is a Gram-positive or Gram-negative bacterial pathogen.

28. A method of inhibiting growth of a pathogen, comprising contacting the pathogen with a composition of claim 14, wherein the composition is provided in an amount effective to inhibit the growth of the pathogen.

29. The method of claim 28, wherein the pathogen of interest is a Gram-positive or Gram-negative bacterial pathogen.
